(19) United States
(12) Patent Application Publication
(10) Pub. No.: US 2024/0392343 A1
(43) Pub. Date: Nov. 28, 2024

(54) METHODS TO SCREEN YEAST TO IDENTIFY XENOBIOTICS THAT TARGET PESTS, PATHOGENS AND PARASITES

(71) Applicants: Peter J. Roy, Toronto (CA); Andrew Robert Burns, Toronto (CA); Jessica Joanne Knox, Hamilton (CA); Brittany Hunter Cooke, Whitby (CA); The Governing Council of the University of Toronto, Toronto (CA)

(72) Inventors: Peter J. Roy, Toronto (CA); Andrew Robert Burns, Toronto (CA); Jessica Joanne Knox, Hamilton (CA); Brittany Hunter Cooke, Whitby (CA)

(21) Appl. No.: 18/673,136

(22) Filed: May 23, 2024

Related U.S. Application Data
(60) Provisional application No. 63/468,415, filed on May 23, 2023.

(30) Foreign Application Priority Data
Jul. 26, 2023 (CA) 3207666

(51) Int. Cl. C12Q 1/18; A01N 43/82; A01P 5/00; C12N 15/10; C12N 15/81; C12Q 1/26

(52) U.S. Cl. CPC C12Q 1/18; A01N 43/82; A01P 5/00; C12N 15/1065; C12N 15/81; C12Q 1/26

(57) ABSTRACT

This disclosure relates to a method to identify xenobiotics that can debilitate a target organism. In particular, the present disclosure provides methods of heterologously expressing a xenobiotic metabolizing enzyme from the target organism in yeast, libraries of yeast strains heterologously expressing a different xenobiotic metabolizing enzyme and methods of screening libraries with xenobiotics to identify a xenobiotic that debilitates a target organism. The disclosure further relates to a method of treating or preventing a nematode infection comprising administering, to a subject in need thereof, an effective amount of a compound of Formula I, or a solvate thereof, as well as certain novel compounds of Formula I, or a solvate thereof.

### RELATED APPLICATION

This application claims the benefit of Canadian Patent Application No. 3,207,666 filed Jul. 26, 2023, which claims the benefit of U.S. Provisional Patent Application Ser. No. 63/468,415 filed May 23, 2023, both of which are incorporated herein by reference in their entirety.

### FIELD

The present disclosure relates to assays to screen yeast to identify xenobiotics that target pests, pathogens and parasites and to libraries of yeast which heterologously express a xenobiotic metabolizing enzyme useful in the screening assays. Nematicides obtained from the screen are also disclosed.

### BACKGROUND

Pests, pathogens and parasites are damaging to crops around the world and can severely limit food production. To overcome this problem, farmers typically apply chemicals to the soil that are lethal to the pests, pathogens and parasites. While these chemicals can be effective at controlling the pests, pathogens and parasites most are also toxic to non-target organisms, such as fish, birds and mammals. The hazards posed to the non-target organisms are so great than many have been withdrawn from the market. Accordingly, new control strategies are needed to improve selectivity for pests, pathogens and parasites while minimizing or eliminating risk to other organisms.

Pathogens and parasites cause millions of human deaths and cause even greater sickness globally every year. For many of these infectious diseases, there are a limited number of effective drugs. For others, the infectious agents have become resistant to current therapies. Accordingly, new control compounds are needed to selectively target pathogens and parasites.

### SUMMARY

The present inventors provide yeast that express a heterologous xenobiotic metabolizing enzyme useful in identifying molecules that selectively debilitate pathogens, parasites and pests of interest and that can be used in a parallel format.

Accordingly, the present disclosure provides an assay to identify xenobiotics that when bioactivated can debilitate a target organism. It further provides libraries of yeast cells which heterologously express xenobiotic metabolizing enzymes and methods of screening the libraries of yeast cells. The present disclosure also provides a method of treating or preventing a nematode infection.

One aspect of the disclosure includes an assay to identify xenobiotics that when bioactivated can debilitate a target organism comprising:
a) heterologously expressing a xenobiotic metabolizing enzyme from the target organism in a yeast;
b) exposing the yeast to a test molecule; and
c) comparing the viability of the yeast in b) with control yeast that do not heterologously express the xenobiotic metabolizing enzyme from the target organism;
wherein if the viability of the yeast is less than the control, then the test molecule is a candidate xenobiotic for debilitating the target organism.

In an embodiment, the xenobiotic metabolizing enzyme is a cytochrome P450 enzyme, an esterase, an alkaline phosphatase, an amidase, a phospholipase, a paraoxonase, a carboxymethylenebutenolidase or a hydrolase from the target organism.

In one embodiment, the xenobiotic metabolizing enzyme is a cytochrome P450 enzyme.

In an embodiment, the coding sequence of the xenobiotic metabolizing enzyme is tagged with a sequence that encodes another protein to enable tracking of the enzyme's expression level and sub-cellular localization.

In one embodiment, the coding sequence of the xenobiotic metabolizing enzyme is codon-optimized.

In an embodiment, the xenobiotic metabolizing enzyme is contained in a plasmid that allows for controlled expression in yeast or is integrated into the yeast genome.

In an embodiment, the yeast comprises a plasmid comprising a unique DNA barcode.

In another embodiment, the yeast comprises a unique DNA barcode integrated into the yeast genome.

In an embodiment, the assay further comprises heterologously expressing xenobiotic metabolizing enzyme co-factors that are required for improved activity of the xenobiotic metabolizing enzyme.

In an embodiment, the xenobiotic metabolizing co-factors are expressed from the same transgene as the xenobiotic metabolizing enzyme.

In another embodiment, the xenobiotic metabolizing co-factors are expressed from a separate transgene than the xenobiotic metabolizing enzyme.

In an embodiment, the target organism is a pest, pathogen or parasite.

In one embodiment, the target organism is a nematode.

In another embodiment, the target organism is an insect.

In an embodiment, the assay further comprises testing the candidate xenobiotic with a yeast heterologously expressing a xenobiotic metabolizing enzyme of a non-target organism, and testing viability of the yeast compared to a control not expressing the xenobiotic metabolizing enzyme of the non-target organism, wherein the candidate xenobiotic is selective against the target organism if the viability of the yeast heterologously expressing the xenobiotic enzyme of the non-target organism is similar to the control.

In an embodiment, the non-target organism is a human, pet, livestock, pollinator, bird, fish, vertebrate or plant.

An aspect includes a library comprising yeast strains, wherein each yeast strain heterologously expresses a different xenobiotic metabolizing enzyme from one or more target organisms, and wherein each yeast strain contains a unique DNA barcode in a plasmid or integrated into a fixed or random location within the yeast genome.

In an embodiment, the xenobiotic metabolizing enzyme in each yeast strain is contained in a plasmid or is integrated into the yeast genome.

In an embodiment, the target organism is a pest, pathogen or parasite.

In one embodiment, the target organism is a nematode.

In another embodiment, the target organism is an insect.

Another aspect includes a library comprising yeast strains, wherein each yeast strain heterologously expresses a different xenobiotic metabolizing enzyme from one or more non-target organisms, wherein each yeast strain contains a unique DNA barcode in a plasmid or integrated into a fixed or random location within the yeast genome.

In an embodiment, the xenobiotic metabolizing enzyme in each yeast strain is contained in a plasmid or is integrated into the yeast genome.

In an embodiment, the non-target organism is a human, pet, livestock, pollinator, bird, fish, vertebrate or plant.

A further aspect includes a method of screening libraries with xenobiotics to identify a xenobiotic that when bioactivated debilitates a target organism, comprising
a) pooling the yeast strains of the library from one or more target organisms as described herein;
b) depositing the pooled library into wells of a multiwell plate;
c) exposing each well of the multiwell plate to a test xenobiotic;
d) incubating the plate of c) to allow the yeast to grow;
e) collecting samples from d) and isolating DNA; and
f) detecting the relative abundance of the unique DNA barcodes;
wherein a decrease in the abundance of a particular unique DNA barcode indicates that the xenobiotic metabolizing enzyme in the yeast strain containing that DNA barcode bioactivates the test xenobiotic and indicates that it is able to debilitate the target organism.

In an embodiment, the method further comprises:
g) pooling the yeast strains of the library from one or more non-target organisms as described herein,
h) depositing the pooled library of g) into wells of a multiwell plate;
i) exposing each well of the multiwell plate to the test xenobiotic;
j) incubating the plate of i) to allow the yeast to grow;
k) collecting samples from j) and isolating DNA; and
l) detecting the relative abundance of the unique DNA barcodes;
wherein the test xenobiotic is selective if it does not decrease the abundance of a particular DNA barcode of the yeast strains of the library of one or more non-target organisms as described herein.

In an embodiment, steps g) to l) are performed in parallel with steps a) to f).

A further aspect includes a method of treating or preventing a nematode infection comprising administering, to a subject in need thereof, an effective amount of a compound of Formula I, or a solvate thereof:

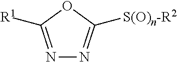

wherein:
R¹ is selected from phenyl and 6-membered heteroaryl, both of which are optionally substituted with one to three substituents independently selected from halo, NO², C₁₋₆alkyl, OC₁₋₆alkyl, C(O)C₁₋₆alkyl, OC(O)C₁₋₆alkyl and C(O)OC₁₋₆alkyl, wherein each alkyl group is optionally fluorosubstituted;
R² is selected from CF₃, C₁₋₆alkyl and C₁₋₆alkenyl; and
n is 0, 1 or 2.

In an embodiment, R¹ is phenyl optionally substituted with one to three substituents independently selected from Cl, Br, F, NO₂, C₁₋₄alkyl, OC₁₋₄alkyl, C(O)C₁₋₄alkyl, OC(O)C₁₋₄alkyl and C(O)OC₁₋₄alkyl, wherein each alkyl group is optionally fluorosubstituted.

In another embodiment, R¹ is phenyl optionally substituted with one to two substituents independently selected from Cl, Br, F, CH₃, CF₃, NO₂, CH₂CH₃, CH(CH₃)₂, C(CH₃)₃, OCH₃, OCF₃, OCH₂CH₃, OCH(CH₃)₂, OC(CH₃)₃, C(O)CH₃, C(O)CF₃, C(O)CH₂CH₃, C(O)CH(CH₃)₂, C(O)C(CH₃)₃, OC(O)CH₃, OC(O)CF₃, OC(O)CH₂CH₃, OC(O)CH(CH₃)₂, OC(O)C(CH₃)₃, C(O)OCH₃, C(O)OCF₃, C(O)OCH₂CH₃, C(O)OCH(CH₃)₂ and C(O)OC(CH₃)₃.

In another embodiment, wherein R¹ is phenyl optionally substituted with one to two substituents independently selected from Cl, Br, F, NO₂, CH₃, CF₃, OCF₃, OCH₃, C(O)CH₃, OC(O)CH₃, and C(O)OCH₃.

In an embodiment, R¹ is phenyl optionally substituted with one to two substituents independently selected from Cl, Br, F, NO₂, CH₃ and C(O)OCH₃.

In an embodiment, R¹ is phenyl substituted with one to two substituents independently selected from Cl and Br.

In an embodiment, R¹ is a 6-membered heteroaryl optionally substituted with one to three substituents independently selected from Cl, Br, F, NO₂, C₁₋₄alkyl, OC₁₋₄alkyl, C(O)C₁₋₄alkyl, OC(O)C₁₋₄alkyl and C(O)OC₁₋₄alkyl.

In an embodiment, R¹ is a 6-membered heteroaryl optionally substituted with one to two substituents independently selected from Cl, Br, F, NO₂, CH₃ and C(O)OCH₃.

In another embodiment, R¹ is an unsubstituted 6-membered heteroaryl.

In one embodiment, the 6-membered heteroaryl is selected from pyridinyl, pyrimidinyl and pyrazinyl.

In another embodiment, the 6-membered heteroaryl is pyridinyl.

In an embodiment, R² is selected from C₁₋₄alkyl and C₁₋₄alkenyl.

In an embodiment, R² is selected from CH₃, CH₂CH₃, CH₂CH₂CH₃ and CH₂CH₂CH₂CH₃.

In another embodiment, R² is selected from CH═CH₂, CH₂C═CH₂, CH₂CH₂C═CH₂ and CH₂CH═CHCH₃.

In one embodiment, R² is CH₂C═CH₂.

In one embodiment, R² is CF₃.

In an embodiment, n is 0.

In an embodiment, the compound of Formula I is selected from:

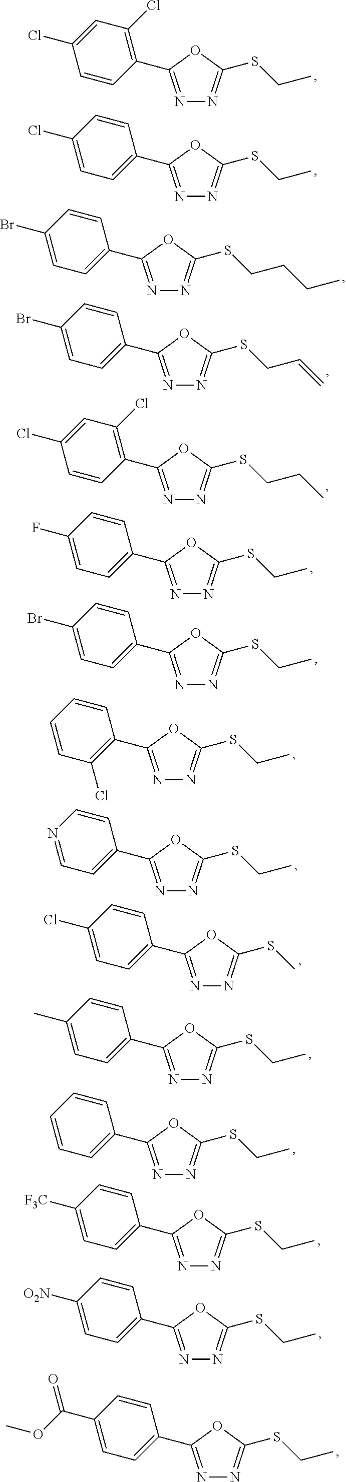

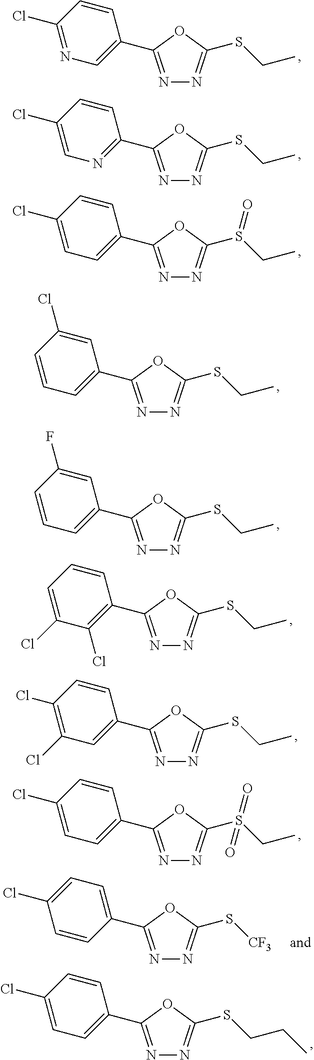

or a solvate thereof.

In an embodiment, the infection is an infection of a nematode of a species selected from members of the genuses Pratylenchus, Heterodera, Meloidogyne, Ditylenchus or Pratylenchus, Cooperia and Haemonchus, including nematodes of the species selected from Heterodera glycines, Globodera pallida, Meloidogyne javanica, Meloidogyne incognita, Meloidogyne arenaria, Radopholus similis, Longidorus elongatus, Meloidogyne hapla, Ditylenchus dipsaci, Pratylenchus penetrans, Cooperia oncophora, and Haemonchus contortus.

In an embodiment, the subject is selected from a human, a mammal, a bird, a plant, a seed, and soil.

In an embodiment, the subject is selected from a plant, a seed and soil and the compound is administered pre-planting, post-planting, as a feed additive, a drench, an external application, a pill and/or by injection.

A further aspect includes a compound selected from:

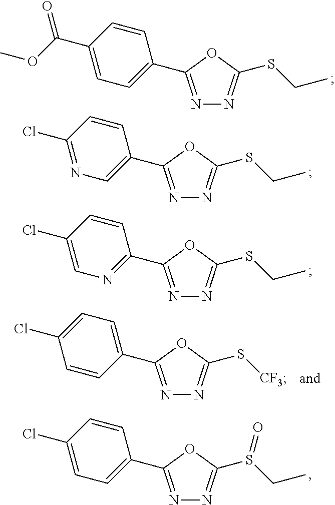

or a solvate thereof.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments but should be given the broadest interpretation consistent with the description as a whole.

### BRIEF DESCRIPTION OF DRAWINGS

The embodiments of the disclosure will now be described in greater detail with reference to the attached drawings in which:

FIG. 1 shows P450-mediated bioactivation of parent molecules into lethal products, in an exemplary embodiment of the disclosure. Selectivin is bioactivated by CYP-35C1 into a highly reactive product that has a non-specific warhead mechanism of action (MOA), meaning that the product disrupts multiple targets at once. Upon generation, reactive warheads bind to products in the cell and are restricted to the cell that generates them.

FIG. 2A-B shows a schematic of the CYPce assay, in an exemplary embodiment of the disclosure. FIG. 2A shows a control yeast strain that does not express a transgenic exogenous P450, which grows at a certain rate in the presence of an exogenous xenobiotic small molecule (molecule X in this example). FIG. 2B shows the experimental yeast strain, which expresses a transgenic exogenous P450 from a targeted organism (called CYP-A in this example), which grows at a certain rate in the presence of molecule X. If the rate of yeast growth of B is significantly lower than that of A, then molecule X is considered a hit. The interpretation is that molecule X is likely metabolized by CYP-A into a lethal product. Follow up experiments are performed to definitively show CYP-mediated metabolism.

FIG. 3A-C shows a schematic of the PEXIL (‘paralleled screens for enzyme-activated xenobiotic-induced lethality) assay, in an exemplary embodiment of the disclosure. FIG. 3A shows a schematic in which each of the two large circles represent the same pool of yeast strains. Each yeast strain (represented by the shapes inside the large circles) expresses one P450 from either humans or a pathogen/parasite. The pool is added to the 96 wells of the plate. Each well contains a unique synthetic small molecule. In this schematic, molecules 40 and 68 specifically kill the strains expressing one P450 from pathogen 1 and 4, respectively. FIG. 3B shows a schematic that illustrates how each strain within the pool has a unique DNA barcode integrated into its genomic DNA. The unique barcodes are flanked by universal common primer sites. The upstream primer has an index sequence that is specific to each well (but is absent from any yeast genome) and the downstream primer has an index sequence that is specific to each plate. In this way, the barcodes from 1000 wells will each be amplified separately, but then combined together to be sequenced in a single next generation sequencing reaction to identify those strains that die in response to specific molecules. It is noted that there are other ways to create DNA indexes to keep track of wells and plates. FIG. 3C shows the bioactivation of bioactivated molecule #1 and #2 by the indicated C. elegans cytochrome P450 when heterologously expressed in uniquely barcoded S. cerevisiae as detected by the PEXIL technology. The S. cerevisiae strains that express the indicated 70 C. elegans cytochrome P450s were pooled and the pools were placed in wells of a 96 well plate. Molecule #1 and molecule #2 were placed in 3 wells each that contained the pools, along with mock-drug treated wells. 48 hours later the pools were individually lysed and the barcodes were amplified and sent for sequencing, where the abundance of each strain in the experimental samples were measured relative to the negative control samples. Molecule #1 was strongly bioactivated by the C. elegans cytochrome P450s CYP-33C2, CP-35A5, CYP-35B2, and CYP-35D1, causing significant reduction in the growth of yeast expressing those P450s in response to the bioactivated molecule #1. Molecule #2 was strongly bioactivated by the C. elegans cytochrome P450s CYP-23A1, CP-34A9, CYP-35A5, and CYP-35C1, causing significant reduction in the growth of yeast expressing those P450s in response to the bioactivated molecule #2.

FIG. 4A-E shows a schematic of the competitive CYPce Assay, in an exemplary embodiment of the disclosure. FIG. 4A shows a control cell/yeast strain that does not express a transgenic exogenous enzyme/P450 grows at a certain rate in the presence of a pre-cidal compound (PCC). FIG. 4B shows a cell/yeast strain that expresses a transgenic exogenous enzyme/P450 of interest that grows at a rate in the presence of the PCC that is significantly slower than the control strain shown in ‘A’.

FIG. 4C shows the background of the sample shown in ‘B’ is used to identify compounds that suppress the slow growth of the enzyme/P450+PCC combination. These ‘suppressing’ molecules will be hits that likely compete with the PCC for the enzyme/P450 and will reveal molecules that also interact with the enzyme/P450. The metabolism of the suppressor hits can be investigated by analytical chemistry techniques (e.g. LCMS). The abundance of the PCC in the presence of the suppressing molecule can be readily assayed by HPLC. FIGS. 4D, E show two examples of PCCs that kill yeast cells in the presence of the heterologously expressed human CYP3A4. Yeast growth was monitored by OD600 readings over 48 hours to produce growth curves depicting growth (Y-axis) over time (X-axis) for each of the 2 PCCs shown. Individual points represent the average reading across replicates (n=2), with error bars representing SEM. Compound identification is represented by its corresponding “AB” number shown in bold at the top of each growth curve.

FIG. 5A-E shows an example of the utility of the CYPce assay that shows the metabolic bioactivation of selectivin-A and structural analogs, in an exemplary embodiment of the disclosure. FIG. 5A shows a schematic of control experiment where yeast that do not express an exogenous P450 are exposed to selectivin-A. In this condition selectivin-A is not metabolized into a lethal product and yeast are viable. FIG. 5B shows a schematic of experimental condition where yeast expressing exogenous CYP-35C1 are exposed to selectivin-A. CYP-35C1 is capable of metabolizing selectivin-A into an active metabolite which leads to reduced viability in the yeast. FIG. 5C shows the selectivin analog dose-response for yeast expressing CYP-35C1 and yeast carrying an empty vector that does not express a P450. Viability relative to untreated controls is indicated by the gray coded scale. FIG. 5D shows the structures of the three selectivin analogs. FIG. 5E shows HPLC-DAD quantification of the three selectivin-A metabolites visible using our HPLC-DAD system (the values of M1, M2 and M3 are combined) in the lysates of yeast expressing CYP-35C1 and empty vector control yeast after treatment with 100 μM selectivin-A. Error bars indicate SEM. The p-values were obtained from unpaired two-tailed Student's t-tests comparing the means of the empty vector control and CYP-35C1 expressing yeast.

FIG. 6A-E shows that selectivins are bioactivated to a toxic electrophile, in an exemplary embodiment of the disclosure. FIGS. 6A-C show mass spectrometry (MS) data for metabolite M1-M3 HPLC fractions and untreated control fractions, as well as MS/MS fragmentation data for selected masses. Structures of γ-glutamylcysteine (γ-Glu-Cys) (A), glutathione (B), and cysteine (C) conjugates of a selectivin-A (sel-A) electrophilic sulfoxide metabolite are shown, along with fragmentations that produce masses found in the MS/MS plots. FIG. 6D shows ¹H and ¹³C NMR characterization of HPLC-purified M2. ¹H-¹H COSY and ¹H-¹³C HMBC correlations are indicated. FIG. 6E shows a schematic of the selectivin-A (sel-A) sulfoxidation and low-molecular-weight-thiol conjugation pathway. S-oxidation produces an electrophilic sulfoxide metabolite that reacts with glutathione (GSH) either spontaneously or via glutathione-S-transferase (GST), and is likely further processed to 7-Glu-Cys and cysteine conjugates via phytochelatin synthase (PCS) and 7-glutamyl transferase (GGT), respectively. Cytochrome p450 enzymes (CYPs) likely mediate S-oxidation.

FIG. 7A-D shows M. incognita CYP4731A3 bioactivates selectivin-A, in an exemplary embodiment of the disclosure. FIG. 7A shows a volcano plot of the yeast-based M. incognita CYP expression screen data. X-axis shows the log₂-transformed fold-difference in growth between empty vector (EV) control and 19 M. incognita CYP-expressing yeast strains resulting from treatment with 50 μM selectivin-A (sel-A). Y-axis shows log₁₀-transformed p-values obtained from unpaired two-tailed Student's t-tests comparing mean relative growth values between CYP-expressing strains and EV control. Each data point corresponds to an individual yeast strain expressing a unique M. incognita CYP enzyme. The data point above the dotted line has a p-value <0.01, and corresponds to the strain expressing M. incognita CYP4731A3. FIG. 7B shows selectivin-A dose-response for yeast expressing CYP4731A3 and yeast carrying empty vector that does not express CYP4731A3. FIG. 7C shows biomass-normalized and background-corrected HPLC chromatograms of lysates from yeast expressing CYP4731A3, as well as empty vector control yeast, treated with 100 μM selectivin-A (sel-A). Unmodified sel-A and metabolites M2 and M3 are indicated. FIG. 7D shows HPLC-DAD quantification of total M2 and M3 in the lysates of yeast expressing M. incognita CYP4731A3 and yeast carrying empty vector that does not express CYP4731A3. AUC is the area under the curve for M2 and M3 combined, calculated at 260 nm. Error bars are SEM. The p-value was obtained from an unpaired one-tailed Student's t-test comparing the means of empty vector control and CYP4731A3 expressing yeast. M1 was not detectable.

FIG. 8A-E shows C. elegans CYP-35D1 bioactivates cyproside-3, in an exemplary embodiment of the disclosure. FIG. 8A shows a schematic of control experiment where yeast that do not express an exogenous P450 are exposed to cyproside-3. In this condition cyproside-3 is not metabolized into a lethal product and yeast are viable. FIG. 8B shows a schematic of experimental condition where yeast expressing exogenous CYP-35D1 are exposed to cyproside-3. CYP-35D1 is capable of metabolizing cyproside-3 into an active metabolite which leads to reduced viability in the yeast. FIG. 8C shows a cyproside-3 dose-response for yeast expressing CYP-35D1 and yeast carrying an empty vector that does not express a P450. Viability relative to untreated controls is indicated by the gray coded scale. FIG. 8D shows HPLC-DAD quantification of the three cyproside-3 metabolites visible using our HPLC-DAD system (M1, M2 and M3) in the lysates of yeast expressing CYP-35D1 and empty vector control yeast after treatment with 100 μM cyproside-3. AUC is the area under the curve, error bars indicate SEM. The p-values were obtained from unpaired two-tailed Student's t-tests comparing the means of the empty vector control and CYP-35D1 expressing yeast. FIG. 8E shows HPLC chromatograms of lysates from yeast expressing CYP-35D1 and empty vector control yeast, treated with 100 μM cyproside-3. Peaks corresponding to the unmodified cyproside-3 and the three cyproside-3 metabolites visible using our HPLC-DAD system (M1, M2 and M3) are indicated.

FIG. 9A-B shows cyproside-3 is bioactivated into a toxic electrophile, in an exemplary embodiment of the disclosure. FIG. 9A shows a schematic of the cyproside-3 sulfoxidation and low-molecular-weight-thiol conjugation pathway. Cytochrome P450 enzymes (CYPs) catalyze the S-oxidation of cyproside-3 which produces an electrophilic sulfoxide metabolite. This sulfoxide metabolite reacts with glutathione (GSH) and is further processed to γ-Glutamyl-Cysteine (γ-Glu-Cys), Cysteinylglycine (Cys-Gly) and cysteine (Cys) conjugates. FIG. 9B shows Mass spectrometry (MS) data for unmodified cyproside-3, the sulfoxide metabolite and the GSH-, γ-Glu-Cys-, Cys-Gly- and Cys-conjugates from lysates of C. elegans treated with cyproside-3 and paired solvent treated controls.

FIG. 10 shows cyproside-3 is a selective nematicide with broad-spectrum activity in plant-parasitic nematodes (PPNs), in an exemplary embodiment of the disclosure. Dose-response analysis of cyproside-3 and tioxazafen on diverse nematode species and off-target systems including human cells (HEK293 and HepG2), fungi (Saccharomyces cerevisiae and Candida albicans) and plant beneficial rhizobacteria (Pseudomonas simiae WCS417 and Pseudomonas defensor WCS374). The activity of the compound relative to the solvent control in each assay is indicated by the gray coded scale.

FIG. 11 shows cyproside-3 is bioactivated by cytochrome P450s in diverse PPN species, in an exemplary embodiment of the disclosure. FIGS. A-A′″ show HPLC chromatograms with cyproside-3 (abbreviated cypros-3) unmodified parent molecule (P) and metabolite (M1-M5) peaks indicated. MP1 is a cyproside-3 metabolite uniquely produced in P. penetrans. Y-axis indicates absorbance wavelength in nm. FIGS. B-B′″ show quantification of unmodified cyproside-3 parent (P) and metabolite peaks from A-A′″. Error bars indicate SEM, p-values calculated using unpaired t-test. FIGS. C-C′″ show the proportion mobile worms relative to DMSO solvent controls after 72-hour exposure to 20 μM cyproside-3 (C. elegans) or 60 μM cyproside-3 (all other species). P-values calculated using unpaired t-test.

FIG. 12A-C shows that the cyproside-3 scaffold controls root infection by M. incognita, in an exemplary embodiment of the disclosure. FIG. 12A shows the structures of cyproside-3 and structural analogs. FIG. 12B shows the number of eggs per milligram of tomato root tissue after 6 weeks of infection in soil drenched with solvent or indicated chemical is reported. Cyprosides 3-2, 3-3, and 3-4 are structural analogs of cyproside-3. FIG. 12C shows the root weight at the infection assay endpoint in each condition. All error bars represent SEM, p-values calculated using an unpaired t test vs. solvent controls. FIGS. 12D, E show that cyproside-3 analogs control root infection by M. incognita, in an exemplary embodiment of the disclosure. FIG. 12D shows the structures of cyproside-3 structural analogs. FIG. 12E shows the number of eggs per milligram of tomato root tissue after 6 weeks of infection in soil drenched with solvent or indicated chemical is reported. Cyprosides 3-7, 3-12, 3-25, and 3-26 are structural analogs of cyproside-3. The other data points represent negative (water, DMSO) and positive (fluopyram, tioxazafen) controls. All error bars represent SEM.

FIG. 13A-D shows M. incognita CYP4731A3 bioactivates cyproside-3, in an exemplary embodiment of the disclosure. FIG. 13A shows a volcano plot of the yeast-based M. incognita CYP expression screen data. The X-axis shows the log 2-transformed fold-difference in growth between empty vector control and 19 M. incognita P450-expressing yeast strains resulting from treatment with 50 μM cyproside-3. Y-axis shows log 10-transformed p-values obtained from unpaired two-tailed Student's t-tests comparing mean relative growth values between CYP-expressing strains and empty vector control. Each data point corresponds to an individual yeast strain expressing a unique M. incognita P450 enzyme. The data point above the dotted line has a p-value <0.01 and corresponds to the strain expressing M. incognita CYP4731A3. FIG. 13B shows the viability of a yeast strain expressing CYP4731A3 and an empty vector control strain after a 30-hour incubation with 50 and 100 μM doses of cyproside-3. Viability relative to untreated controls is indicated by the grey-coded scale. FIG. 13C shows HPLC chromatograms of lysates from yeast expressing CYP4731A3 and empty vector control yeast, treated with 100 μM cyproside-3. Peaks corresponding to the unmodified cyproside-3 and the three cyproside-3 metabolites visible using the HPLC-DAD system (M1, M2 and M3) are indicated. FIG. 13D shows HPLC-DAD quantification of the M1, M2 and M3 metabolites in the lysates of yeast expressing CYP4731A3 and empty vector control yeast. AUC is the area under the curve, error bars indicate SEM. The p-values were obtained from unpaired two-tailed Student's t-tests comparing the means of the empty vector control and CYP4731A3 expressing yeast.

FIG. 14A-B shows CYPce is used to screen 3000 small molecules for those that can be bioactivated to lethal metabolites by mosquito (A. gambiae) P450s, in an exemplary embodiment of the disclosure. Two unique mosquito P450-expressing yeast strains were treated with 3,000 compounds from a custom AB3000 library in parallel with an empty vector control strain. The treated and untreated (DMSO) growth was monitored over 48 hours by OD600 absorbance readings. FIG. 14A shows the normalized area under the curve (AUC) of yeast expressing A. gambiae CYP6M2 treated with each AB3000 compound relative to the control strain. Each point represents the average treated AUC of the P450-expressing yeast strain (n=2) normalized to its own untreated controls (n=64), relative to the treated AUC of the control strain (n=2) normalized to its own untreated controls (n=64). The log 10 transformed relative AUC is shown on the Y-axis, with the respective AB3000 compound names shown on the X-axis. Clear points outlined in black represent cases where the compound was too toxic to the control strain to be defined as a hit (<0.3 normalized AUC). Points in black represent hits defined by the stringency parameters. FIG. 14B shows the normalized AUC of yeast expressing A. gambiae CYP9J5 treated with each AB3000 compound relative to the control strain. Points in black represent hits defined by the stringency parameters. FIG. 14C shows the normalized AUC of yeast expressing either A. gambiae CYP6M2, A. gambiae CYP9J5, or human CYP3A4 treated with each of the 44 mosquito hits identified in the screen of the AB3000 library. The intensity of the black coloured boxes corresponds to lethality observed in response to treatment with the indicated compound. Compounds for which there is no CYP3A4 data available are hashed. All strains, including the control strain were normalized to the treated AUC of the control strain, showing lethality due to P450-dependent activity alone. The asterisks indicates that the molecule impacts yeast expressing human CYP3A4 by less than 30% relative to the control strain.

FIG. 15 shows the performance of 44 hits from the screen of the AB3000 library against yeast expressing either A. gambiae CYP6M2 or CYP9J5, in an exemplary embodiment of the disclosure. The AB3000 library was screened in duplicate against yeast strains expressing either A. gambiae CYP9J5, A. gambiae CYP9J5, or an empty vector control. Growth was monitored by OD600 readings over 48 hours to produce growth curves depicting growth (Y-axis) over time (X-axis) for each of the 44 hits. Individual points represent the average reading across replicates (n=2), with error bars representing SEM. Growth data of yeast expressing human cyp3a4 is shown for hits AB1008-2025. Compound identification is represented by its corresponding “AB” number shown in bold at the top of each growth curve.

FIG. 16 shows the performance of 7 hits from the screen of the 1,280 AB3000 library compounds identified 7 hits against yeast expressing human cyp3a4, in an exemplary embodiment of the disclosure. 1,280 AB3000 compounds (AB1001-2280) were screened in duplicate against yeast expressing either human CYP3A4 or an empty vector control. Growth was monitored by OD600 readings over 48 hours to produce growth curves depicting growth (Y-axis) over time (X-axis) for each of the 7 hits (two of the hits are shown in FIG. 4). Individual points represent the average reading across replicates (n=2), with error bars representing SEM. Compound identification is represented by its corresponding “AB” number shown in bold at the top of each growth curve.

FIG. 17 shows cyproside-3 parent (Cyproside-3-1) and 20 structural analogs with R¹, R² and (O)ₙ features as defined tested at the indicated concentrations (μM) for nematicidal activity in free-living nematode C. elegans and plant-parasitic nematode species D. dipsaci and M. hapla. In C. elegans the number of living worms after 5 days of compound exposure is indicated by the grey-scale coded scale. In D. dipsaci and M. hapla the proportion of mobile worms in each condition relative to the DMSO solvent control after 5 days of compound exposure is indicated by the grey-scale legend. These summary data are the average of 3 technical replicates.

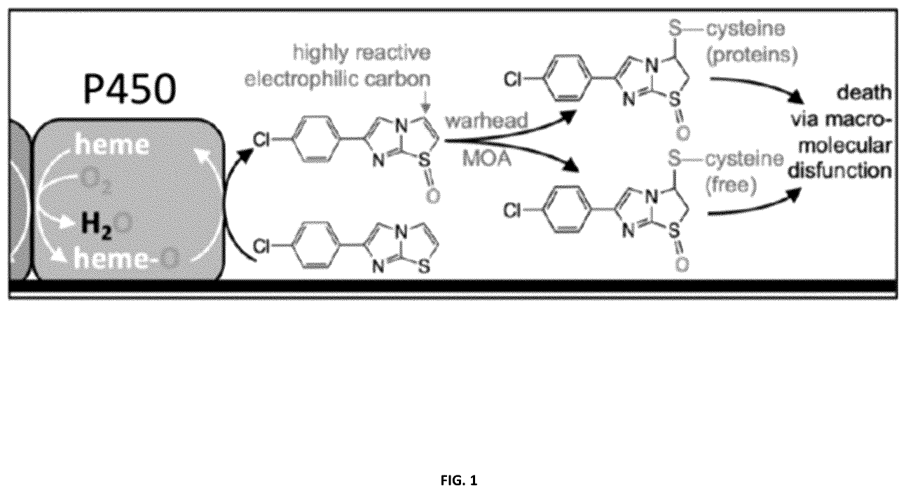

### DETAILED DESCRIPTION

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature described herein may be combined with any other feature or features described herein.

### I. Definitions

Unless otherwise indicated, the definitions and embodiments described in this and other sections are intended to be applicable to all embodiments and aspects of the present disclosure herein described for which they are suitable as would be understood by a person skilled in the art.

As used herein, the following terms may have meanings ascribed to them below, unless specified otherwise. However, it should be understood that other meanings that are known or understood by those having ordinary skill in the art are also possible, and within the scope of the present disclosure. All publications, patent applications, patents, and other references mentioned herein are incorporated by reference in their entirety. In the case of conflict, the present specification, including definitions, will control. In addition, the materials, methods, and examples are illustrative only and not intended to be limiting.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this disclosure, the singular forms “a”, “an” and “the” include plural references unless the content clearly dictates otherwise.

In embodiments comprising an “additional” or “second” component, the second component as used herein is chemically different from the other components or first component. A “third” component is different from the other, first, and second components, and further enumerated or “additional” components are similarly different.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

It should also be understood that, in certain methods described herein that include more than one step or act, the order of the steps or acts of the method is not necessarily limited to the order in which the steps or acts of the method are recited unless the context indicates otherwise.

The term “nucleic acid” as used herein may refer to a biopolymer comprising monomers of nucleotides, such as deoxyribonucleic acid (DNA), ribonucleic acid (RNA) and other polynucleotides of modified nucleotides and/or nucleotide derivatives, and may be either double stranded (ds) or single stranded (ss). “Modified” bases include, for example, tritiated bases and unusual bases such as inosine. A variety of modifications can be made to DNA and RNA; thus “nucleic acid molecule”, “DNA molecule”, and “RNA molecule” embrace chemically, enzymatically, or metabolically modified forms. Examples of modified nucleotides which can be used to generate the nucleic acids disclosed herein include xanthine, hypoxanthine, 2-aminoadenine, 6-methyl, 2-propyl and other alkyl adenines, 5-halo uracil, 5-halo cytosine, 6-aza uracil, 6-aza cytosine and 6-aza thymine, pseudo uracil, 4-thiouracil, 8-halo adenine, 8-aminoadenine, 8-thiol adenine, 8-thiolalkyl adenines, 8-hydroxyl adenine and other 8-substituted adenines, 8-halo guanines, 8 amino guanine, 8-thiol guanine, 8-thiolalkyl guanines, 8-hydroxyl guanine and other 8-substituted guanines, other aza and deaza uracils, thymidines, cytosines, adenines, or guanines, 5-trifluoromethyl uracil and 5-trifluoro cytosine or fluorophore and quencher conjugated nucleotides. Alternatively, the nucleic acid molecules can be produced biologically using an expression vector. In some embodiments, modified nucleotides comprise one or more modified bases (e.g. unusual bases such as inosine, and functional modifications to the bases such as amino modifications), modified backbones (e.g. peptide nucleic acid, PNA) and/or other chemically, enzymatically, or metabolically modified forms. The term “functional fragment” as used herein refers to a fragment of the nucleic acid that retains the functional property of the full-length nucleic acid, for example, for a xenobiotic metabolizing enzyme, the ability to metabolize a xenobiotic. In some embodiments, modified nucleotides may contain one or more modified bases (e.g. unusual bases such as inosine, and functional modifications to the bases such as amino), modified backbones (e.g. peptide nucleic acid, PNA) and/or other chemically, enzymatically, or metabolically modified forms.

The term “polypeptide” or “protein” means a molecule that comprises at least two amino acid residues linked by a peptide bond to form a polypeptide. Small polypeptides of less than about 50 amino acids may be referred to as “peptides”.

The terms “vector” and “plasmid” are used herein interchangeably and refer to DNA that is contained in a cell. The DNA contained in the cell may be stably expressed or integrated into the genome of the cell.

The term “DNA barcode” as used herein refers to a unique string of DNA bases that are flanked by common PCR primer binding sites common to all of the DNA barcodes. The DNA barcodes can be amplified by PCR and sequenced. The presence or absence of a particular DNA barcode can be used to identify which xenobiotic metabolizing enzymes can metabolize a candidate xenobiotic.

The term “xenobiotic” as used herein refers to any molecule that is foreign to a given target organism.

The term “heterologous” as used herein refers to a nucleic acid sequence or polypeptide sequence that has been derived from a different cell type or different species than the cell in which it is in. For example, in a yeast cell, a heterologous sequence is a nucleic acid sequence or polypeptide sequence not naturally occurring in yeast.

The term “bioactivate” as used herein refers to the enzymatic metabolism of a compound into an active compound, such as a toxic compound. In the context of the present disclosure, bioactivation refers to the metabolism of a xenobiotic into a harmful product which debilitates a target organism. In some embodiments, the compound is bioactivated to produce highly reactive non-specific products that disrupt multiple targets at once.

The term “target organism” as used herein refers to an organism for which debilitating xenobiotics are being assayed.

The term “debilitate” as used herein refers to damaging or killing the cell or organism. In an embodiment, debilitating may refer to damaging a cell, wherein the cell is damaged 99%, 95%, 90%, 80%, 70%, 60%, 50%, 40%, 30%, 20%, 10% or less than 10% compared to a non-damaged cell. In an embodiment, killing refers to inducing cell death through any of a variety of mechanisms including apoptosis, necrosis and autophagy. For example, an agent that is cytotoxic kills the cells.

The term “viability” as used herein refers to the ability of the yeast to grow in culture. For example, the yeast may be 100%, 99%, 95%, 90%, 80%, 70%, 60%, 50%, 40%, 30%, 20%, 10% or 0% viable as compared to a control. A person skilled in the art can readily test for viability, for example, by measuring optical density.

The term “test molecule” as used herein refers to a molecule, compound, or chemical applied to the yeast to determine if the test molecule is a candidate debilitating xenobiotic.

The term “cytochrome P450 enzyme” or “CYP P450” or “P450” or “CYP” as used herein refers to a class of enzymes that oxidize substances using iron and can metabolize xenobiotics.

The term “esterase” as used herein refers to a class of enzymes that catalyze the hydrolysis of esters.

The term “alkaline phosphatase” as used herein refers to a class of enzymes that catalyze the hydrolysis of phosphate esters.

The term “amidase” as used herein refers to a class of enzymes that catalyze the hydrolysis of amides.

The term “phospholipase” as used herein refers to a class of enzymes that catalyze the hydrolysis of phospholipids.

The term “paraoxonase” as used herein refers to a class of enzymes that catalyze the hydrolysis of organophosphates. Paraoxonases include the isoenzymes paraoxonase 1, paraoxonase 2 and paraoxonase 3.

The term “carboxymethylenebutenolidase” refers to a class of enzymes that catalyze the hydrolysis of carboxylic ester bonds.

The term “hydrolase” as used herein refers to a class of enzymes that catalyze biochemical reactions using water to break chemical bonds.

The term “xenobiotic metabolizing enzyme co-factors” as used herein refers to additional factors, derived from the same target organism, which facilitate optimal activity of the xenobiotic-metabolizing enzyme.

The term “codon-optimized” as used herein refers to changes in the sequence of the xenobiotic metabolizing enzyme, i.e., changing the codons that code for the same amino acid residues, in order to optimize expression.

The term “library” as used herein refers to a collection of cells or organisms. For example, in the context of the present application the cells or organisms may express different xenobiotic metabolizing enzymes and/or sequence variations, such as a mutation, of the same xenobiotic metabolizing enzymes.

The term “solvate” as used herein means a compound, or a salt or prodrug of a compound, wherein molecules of a suitable solvent are incorporated in the crystal lattice. A suitable solvent is physiologically tolerable at the dosage administered.

The term “alkyl” as used herein means straight or branched chain, saturated alkyl groups. The number of carbon atoms that are possible in the referenced alkyl group are indicated by the prefix “Cₙ₁₋ₙ₂”. Thus, for example, the term “C₁₋₆alkyl” (or “C₁-C₆alkyl”) means an alkyl group having 1, 2, 3, 4, 5, or 6 carbon atoms and includes, for example, any of the hexyl alkyl and pentyl alkyl isomers as well as n-, iso-, sec- and tert-butyl, n- and iso-propyl, ethyl and methyl. As another example, “C₄alkyl” refers to n-, iso-, sec- and tert-butyl, n- and isopropyl, ethyl and methyl.

The term “alkenyl” as used herein means straight or branched chain, unsaturated alkenyl groups containing at least one double bond. The number of carbon atoms that are possible in the referenced alkenyl group are indicated by the prefix “Cₙ₁₋ₙ₂”. For example, the term C₂₋₆alkynyl means an alkynyl group having 2, 3, 4, 5 or 6 carbon atoms.

The term “heteroaryl” as used herein refers to heteroaromatic ring containing 5-6 atoms in which one or more of the atoms are a heteroatom selected from O, S and N and the remaining atoms are C.

The term “halogen” (or “halo”) whether it is used alone or as part of another group, refers to a halogen atom and includes fluoro, chloro, bromo and iodo.

The term “fluoro-substituted” as used herein means that one or more of the available hydrogen atoms in a referenced group have been replaced with a fluorine atom.

The term “available”, as in “available hydrogen atoms” or “available atoms” refers to atoms that would be known to a person skilled in the art to be capable of replacement by a substituent.

The term “pharmaceutically acceptable” means compatible with the treatment of subjects.

The term “nematode” as used herein refers to a worm of the phylum Nematoda.

The term “nematode infection” as used herein refers to an invasion of any part of a subject by a foreign undesirable nematode.

The term “nematicidal composition” as used herein refers to a composition of matter for treating one or more nematode infections.

As used herein, the term “effective amount” or “therapeutically effective amount” means an amount effective, at dosages and for periods of time necessary to achieve a desired result. For example, in the context of treating a nematode infection, an effective amount of a compound is an amount that, for example, reduces the nematode infection compared to the nematode infection without administration of the compound. By “reducing the infection”, it is meant, for example, reducing the amount of the infectious agent in the subject and/or reducing the symptoms of the infection. The amount of a given compound or composition that will correspond to such an amount will vary depending upon various factors, such as the given compound or composition, the formulation, the route of administration, the type of condition, disease or disorder, the identity of the subject being treated, and the like, but can nevertheless be routinely determined by one skilled in the art.

The terms “to treat”, “treating” and “treatment” as used herein and as is well understood in the art, means an approach for obtaining beneficial or desired results, including clinical results. Beneficial or desired clinical results include, but are not limited to, diminishment of extent of nematode infection, stabilization (i.e. not worsening) of the state of the nematode infection, preventing spread of the nematode infection, delay or slowing of infection progression, amelioration or palliation of the nematode infectious state, diminishment of the reoccurrence of nematode infection, diminishment, stabilization, alleviation or amelioration of one or more diseases, disorders or conditions arising from the nematode infection, diminishment of the reoccurrence of one or more diseases, disorders or conditions arising from the nematode infection, and remission of the nematode infection and/or one or more symptoms or conditions arising from the nematode infection, whether partial or total, whether detectable or undetectable. “To treat”, “treating” and “treatment” can also mean prolonging survival as compared to expected survival if not receiving treatment. “To treat”, “treating” and “treatment” as used herein also include prophylactic treatment. For example, a subject with an early nematode infection is treated to prevent progression, or alternatively a subject in remission is treated to prevent recurrence.

“Palliating” an infection, disease, disorder and/or condition means that the extent and/or undesirable manifestations of an infection, disease, disorder and/or condition are lessened and/or time course of the progression is slowed or lengthened, as compared to not treating the infection, disease, disorder and/or condition.

The term “prevention” or “prophylaxis” and the like as used herein refers to a reduction in the risk or probability of a plant becoming afflicted with a nematode infection and/or a disease, disorder and/or condition arising from a nematode infection or manifesting a symptom associated with a nematode infection and/or a disease, disorder and/or condition arising from a nematode infection.

The recitation of numerical ranges by endpoints herein includes all numbers and fractions subsumed within that range (e.g. 1 to 5 includes for example 1, 1.5, 2, 2.75, 3, 3.90, 4, and 5). It is also to be understood that all numbers and fractions thereof are presumed to be modified by the term “about”.

It will be understood that any component defined herein as being included can be explicitly excluded by way of proviso or negative limitation, such as any specific compounds or method steps, whether implicitly or explicitly defined herein.

### II. Assays, Methods and Uses

Disclosed herein are assays to identify xenobiotics that when bioativated can debilitate a target organism. The present inventors have shown that yeast cells can heterologously express a xenobiotic metabolizing enzyme from the target organism to identify candidate xenobiotics. The term “CYPce assay” as used herein refers to this assay when the assay comprises a S. cerevisiae yeast cell that utilizes a CYP P450 enzyme.

Accordingly, an aspect of the present disclosure is an assay to identify xenobiotics that when bioactivated can debilitate a target organism comprising (a) heterologously expressing a xenobiotic metabolizing enzyme from the target organism in a yeast; (b) exposing the yeast to a test molecule; and (c) comparing the viability of the yeast in (b) with control yeast that do not heterologously express the xenobiotic metabolizing enzyme from the target organism; wherein if the viability of the yeast is less than the control, then the test molecule is a candidate xenobiotic for debilitating the target organism.

Yeast strains may include, without limitation, genera and species from the kingdom Fungi. In one embodiment, the species are, without limitation, Aspergillus acidus, Aspergillus niger, Aspergillus oryzae, Aspergillus sojae, Candida etchellsii, Candida milleri, Candida oleophila, Candida rugosa, Candida tropicalis, Candida versatilis, Candida zemplinina, Candida zeylanoides, Cyberlindnera jadinii, Cyberlindnera mrakii, Cystofilobasidium infirmominiatum, Debaryomyces hansenii, Dekkera bruxellensis, Fusarium domesticum, Fusarium venenatum, Galactomyces candidum, Geotrichum candidum, Guehomyces pullulans, Hanseniaspora guilliermondii, Hanseniaspora osmophila, Hanseniaspora uvarum, Kazachstania exigua, Kazachstania unispora, Kluyveromyces lactis, Kluyveromyces marxianus, Lachancea fermentati, Lachancea thermotolerans, Lecanicillium lecanii, Metschnikowia pulcherrima, Mucor hiemalis, Mucor mucedo, Mucor plumbeus, Mucor racemosus, Neurospora sitophila, Penicillium camemberti, Penicillium caseifulvum, Penicillium chrysogenum, Penicillium commune, Penicillium nalgiovense, Penicillium roqueforti, Penicillium solitum, Pichia fermentans, Pichia kluyveri, Pichia kudriavzevii, Pichia membranfaciens, Pichia occidentalis, Pichia pijperi, Rhizopus microspores, Rhizopus oligosporus, Rhizopus oryzae, Rhizopus stolonfer, Saccharomyces bayanus, Saccharomyces cerevisiae, Schizosaccharomyces pombe, Schwanniomyces vanrijiae, Scopulariopsis flava, Starmerella bombicola, Torulaspora delbrueckii, Torulopsis candida, Torulopsis holmii, Trigonopsis cantarellii, Wickerhamomyces anomalus, Yarrowia lipolytica, Zygosaccharomyces rouxii, Zygotorulaspora florentina. There are a variety of commercial sources for yeast strains, such as Lallemand Inc. (Canada), AB Mauri (Australia) and Lesaffre (France).

In one embodiment the yeast is from Saccharomyces. In a particular embodiment, the yeast is Saccharomyces cerevisiae.

In some embodiments, the assay is conducted in yeast cells; in other embodiments, the assay is conducted in other cell types, including, but not limited to, cultured human cells.

The xenobiotic metabolizing enzyme may be any enzyme that is capable of bioactivating a pro-molecule into an active molecule. In an embodiment, the xenobiotic metabolizing enzyme is a cytochrome P450 enzyme, an esterase, an alkaline phosphatase, an amidase, a phospholipase, a paraoxonase, a carboxymethylenebutenolidase or a hydrolase from the target organism.

In one embodiment, the xenobiotic metabolizing enzyme is a cytochrome P450 enzyme.

In one embodiment, the cytochrome P450 enzyme is human CYP, such as CYP3A4. In an embodiment, the sequence of CYP3A4 is found at Genbank Gene ID: 1576 or UniProt P08684.

In another embodiment, the cytochrome P450 enzyme is C. elegans CYP, such as CYP-35C1. In an embodiment, the sequence of CYP-35C1 is found at Genbank Gene ID: 179885 or UniProt G5ECD0. In another embodiment, the cytochrome P450 enzyme is C. elegans CYP-35D1. In an embodiment, the sequence of CYP-35D1 is found at UniProt 045364.

In yet another embodiment, the cytochrome P450 enzyme is Meloidogyne incognita CYP, such as CYP4731A3. In one embodiment, the sequence of CYP4731A3 is found at Wormbase ParaSite BioMart Minc3s00939g19098.

In another embodiment, the cytochrome P450 enzyme is mosquito, such as CYP6M2. In one embodiment, the sequence of CYP6M2 is found at Vectorbase AGAP008212. In another embodiment, the cytochrome P450 enzyme is mosquito CYP9J5. In one embodiment, the sequence of CYP9J5 is found at Vectorbase AGAP012296.

In some embodiments, the cytochrome P450 enzyme is from a mosquito species or a tick species and the target organism is the mosquito species or tick species from which the cytochrome P450 is derived. In some embodiments, the cytochrome P450 enzyme is an enzyme in Table 1, the target organism is the target organism from which the cytochrome P450 is derived as listed in Table 1 and the accession number for the cytochrome P450 sequence is also listed in Table 1.

In an embodiment, the coding sequence of the xenobiotic metabolizing enzyme is tagged with a sequence that encodes another protein to enable tracking of the enzyme's expression level and sub-cellular localization, for example, green fluorescent protein (GFP) or variants thereof, or epitope tags such as FLAG, MYC, or HIS.

In one embodiment, the coding sequence of the xenobiotic metabolizing enzyme is codon-optimized. A person skilled in the art would readily understand that codon optimization refers to improvements which change the codon composition of a gene without altering the amino acid sequence. This is possible due to the redundancy of the genetic code.

In an embodiment, the xenobiotic metabolizing enzyme is contained in a plasmid that allows for controlled expression in yeast or is integrated into the yeast genome.

In an embodiment, the yeast comprises a plasmid comprising a unique DNA barcode.

In another embodiment, the yeast comprises a unique DNA barcode integrated into the yeast genome.

In an embodiment, the assay further comprises heterologously expressing xenobiotic metabolizing enzyme co-factors that are required for improved activity of the xenobiotic metabolizing enzyme.

In one embodiment the xenobiotic metabolizing enzyme co-factors are P450 oxidoreductase and/or cytochrome b5.

In an embodiment, the xenobiotic metabolizing co-factors are expressed from the same transgene as the xenobiotic metabolizing enzyme.

In another embodiment, the xenobiotic metabolizing co-factors are expressed from a separate transgene than the xenobiotic metabolizing enzyme.

The target organism can be any organism or cell of an organism other than yeast or a yeast cell that is desired to be inhibited or killed. In an embodiment, the target organism is a pest, pathogen or parasite. In another embodiment, the target organism or cell is a cancerous cell or a disease vector that over-expresses P450s.

In one embodiment, the target organism is a nematode. In one embodiment, the nematode is C. elegans.

In some embodiments, the nematode is a nematode of the genus Meloidogyne.

In another embodiment, the nematode is Meloidogyne incognita.

In some embodiments, the nematode is a nematode of the following non-limiting, exemplary genera: Caenorhabditis, Nippostrongyles, Anguina, Ditylenchus, Tylenchorhynchus, Pratylenchus, Radopholus, Hirschmanniella, Nacobbus, Hoplolaimus, Scutellonema, Rotylenchus, Helicotylenchus, Rotylenchulus, Belonolaimus, Heterodera, other cyst nematodes, Meloidogyne, Criconemoides, Hemicycliophora, Paratylenchus, Tylenchulus, Aphelenchoides, Bursaphelenchus, Rhadinaphelenchus, Longidorus, Xiphinema, Trichodorus, Paratrichodorus, Dirofiliaria, Onchocerca, Brugia, Acanthocheilonema, Aelurostrongylus, Anchlostoma, Angiostrongylus, Ascaris, Bunostomum, Capillaria, Chabertia, Cooperia, Crenosoma, Dictyocaulus, Dioctophyme, Dipetalonema, Dracunculus, Enterobius, Filaroides, Haemonchus, Lagochilascaris, Loa, Manseonella, Muellerius, Necator, Nematodirus, Oesophagostomum, Ostertagia, Parafilaria, Parascaris, Physaloptera, Protostrongylus, Setaria, Spirocerca, Stephanogilaria, Strongyloides, Strongylus, Thelazia, Toxascaris, Toxocara, Trichinella, Trichostrongylus, Trichuris, Uncinaria or Wuchereria.

In some embodiments, the nematodes are of the genera Cooperia, Haemonchus, Caenorhabditis, Nippostrongyles, Dirofilaria, Onchocerca, Brugia, Acanthocheilonema, Dipetalonema, Loa, Mansonella, Parafilaria, Setaria, Stephanofilaria, Wucheria, Pratylenchus, Heterodera, Meloidogyne, Ditylenchus or Paratylenchus. In some embodiments the nematodes are of the species Cooperia oncophora, Haemonchus contortus, Caenorhabditis elegans, Nippostrongyles brasiliensis, Ancylostoma caninum, Trichinella spiralis, Trichuris muris, Dirofilaria immitis, Dirofilaria tenuis, Dirofilaria repens, Dirofilaria ursi, Ascaris suum, Toxocara canis, Toxocara cati, Strongyloides ratti, Parastrongyloides trichosuri, Heterodera glycines, Globodera pallida, Meloidogyne javanica, Meloidogyne incognita, Meloidogyne arenaria, Radopholus similis, Longidorus elongatus, Meloidogyne hapla, Ditylenchus dipsaci or Pratylenchus penetrans.

In some embodiments, the target organism is a helminth.

In some embodiments, the helminth is a helminth of the following non-limiting, exemplary genera: Taenia solium, Taenia saginata, Taenia asiatica, Schistosoma species, including Schistosoma haematobium and Schistosoma mansoni. Echinococcus granulosus, Clonorchis sinensis, Paragonimus westermani, Opisthorchis viverrini, Opisthorchis felineus, Cyclospora cayetanensis.

In another embodiment, the target organism is an insect. In one embodiment, the insect is mosquito.

In another embodiment, the target organism is an insect. In one embodiment, the insect is mosquito, including Anopheles gambiae, Anopheles freeborni, Anopheles quadrimaculatus, Anopheles atroparvus, Anopheles labranchiae, Anopheles plumeus, Anolpheles sacharovi, Aedes aegypti, Aedes albopictus, Aedes atropalpus, Aedes japonicus, Aedes koreicus, Aedes triseriatus, Culex pipiens, Culex tarsalis, and Culex quinquefasciatus. In another embodiment, the insect is lyme disease-transmitting tick, including Ixodes ricinus and Ixodes scapularis. In yet another embodiment, the insect is Cimex lectularius, Tunga penetrans, Sarcoptes scabiei var hominis, Phlebotomus papatasi, Lutzomyia longipalpis, Pediculus humanus corporis

In one embodiment, the insect is any of the following non-limiting, exemplary genera and species: Schistocerca gregaria, Aphis fabae, Anabrus simplex, Popillia japonica, Manduca quinquemaculata, Anoplocnemis curvipes, Nephotettix nigropictus, Cosmopolites sordidus, Xylotrechus quadripes, Diabrotica virgifera, Halyomorpha halys, Haplothrips aculeatus, Leptinotarsa decemlineata, Pectinophora gossypiella, Anasa tristis, Cydia pomonella, Thaumatotibia leucotreta, Pectinophora gossypiella, Teia anartoides, Cactoblastisl cactorun, Ectomyelois caratonia, Plutella xylostella, Lymantria ninayi, L. rosa, L. novaguinensis, Calliteara queenslandica, Dasychira wandammena, Milionia isodoxa, Alcis papuensis, Paradromulia nigrocellata, Agrotis ipsilon, Hyposidera talcata, Acacia mangium, the genus Syntherata spp, Striglina floccosa (Thyrididae) and Scopelodes venosa (Limacodidae), Hypsipyla robusta, Hyblaea puera, the genera Eumeta, including Eurema blanda, the genera Hyalarcta, Aeropedellus clavatus, Ageneotettix deorum, Amphitornus coloradus, Aulocara elliotti, Aulocarafemoratum, Boopedon nubilum, Cordillacris crenulate, Cordillacris occipitalis, Eritettix simplex, Opeia obscura, Phlibostroma quadrimaculatum, Psoloessa delicatula, Arphia pseudonietana, Camnula pellucida, Hadrotettix trifasciatus, Mestobregma plattei, Metator pardalinus, Spharagemon equale, Trachyrhachys kiowa, Trimerotropis pallidipennis, Xanthippus corallipes, Hesperotettix viridis, Melanoplus bivittatus, Melanoplus confuses, Melanoplus cuneatus, Melanoplus foedus, Melanoplus gladstoni, Melanoplus infantilis, Melanoplus keeleri, Melanoplus occidentalis, Melanoplus sanguinipes, Selatosomus destructor (previously Ctenicera/aeripennis destructor), Ctenicera lobata, Ctenicera morula, Athous spp, Limonius canus, Limonius californicus, Hypnoides spp., including Hypnoidus bicolor, Aeolus mellillus, Cephus cinctus, Sitodiplosis mosellana, Sitona lineatus, Plutella maculipennis, Ceutorhynchus obstrictus, Mamestra configurata, Anoplophora glabripennis, members of the Tenthredinidae family, Tetropium fuscum, Ophiognomonia clavigignenti-juglandacearum, Agrilus planipennis, Malacosoma disstria, Dendroctonus ponderosae, Choristoneura fumiferana, Choristoneurafreeman.

In another embodiment, the target organism is a fungus. In one embodiment, the organism is a fungus of any of the following non-limiting, exemplary genera and species: Histoplasma capsulatum, Cryptococcus neoformans, Cryptococcus gattii, Coccidioides immitis, Coccidioides posadasii, Candidaa species, including Candida albicans, Candida tropicalis, Candida auris, Candida parapsilosis, Candida krusei, Candida glabrata, Candida lusitaniae, Aspergillus flavus, Aspergillus fumigatus, Magnaporthe grisea, Histoplasma spp., eumycetoma causative agents, Mucorales, Fusarium spp., Candida tropicalis, Candida parapsilosis, Scedosporium spp., Lomentospora prolificans, Coccidioides spp., Talaromyces marneffei, Pneumocystis jirovecii, Paracoccidioides spp.

In another embodiment, the target organism is a protazoan. In one embodiment, the organism is a protozoan of any of the following non-limiting, exemplary genera and species: Mycobacterium tuberculosis, Pseudomonas aeruginosa, Trypanosoma brucei gambiense, Toxoplasma gondii, Entamoeba histolytica, Giardia duodenalis, Giardia lamblia, Cryptosporidium parvum, Naegleria fowleri.

In an embodiment, the assay further comprises testing the candidate xenobiotic with a yeast heterologously expressing a xenobiotic metabolizing enzyme of a non-target organism, and testing viability of the yeast compared to a control not expressing the xenobiotic metabolizing enzyme of the non-target organism, wherein the candidate xenobiotic is selective against the target organism if the viability of the yeast heterologously expressing the xenobiotic enzyme of the non-target organism is similar to the control.

In an embodiment, the non-target organism is a human, pet, livestock, pollinator, bird, fish, vertebrate or plant.

Another aspect of the disclosure is a library comprising yeast strains, wherein each yeast strain heterologously expresses a different xenobiotic metabolizing enzyme from one or more target organisms, and wherein each yeast strain contains a unique DNA barcode in a plasmid or integrated into a fixed or random location within the yeast genome.

In an embodiment, the xenobiotic metabolizing enzyme in each yeast strain is contained in a plasmid or is integrated into the yeast genome.

The target organism can be any organism or cell of an organism other than yeast or a yeast cell that is desired to be inhibited or killed. In an embodiment, the target organism is a pest, pathogen or parasite. In another embodiment, the target organism or cell is a cancerous cell or a disease vector that over-expresses P450s.

In an embodiment, the target organism is as described herein.

In one embodiment, the target organism is a nematode. In one embodiment, the nematode is C. elegans. In another embodiment, the nematode is Meloidogyne incognita.

In another embodiment, the target organism is an insect. In one embodiment, the insect is mosquito.

Another aspect of the disclosure is a library comprising yeast strains, wherein each yeast strain heterologously expresses a different xenobiotic metabolizing enzyme from one or more non-target organisms, wherein each yeast strain contains a unique DNA barcode in a plasmid or integrated into a fixed or random location within the yeast genome.

In an embodiment, the xenobiotic metabolizing enzyme in each yeast strain is contained in a plasmid or is integrated into the yeast genome.

In an embodiment, the non-target organism is a human, pet, livestock, pollinator, bird, fish, vertebrate or plant.

Also provided is a method of screening libraries with xenobiotics to identify a xenobiotic that when bioactivated debilitates a target organism comprising (a) pooling the yeast strains of the library from the one or more target organisms; (b) depositing the pooled library into wells of a multiwell plate; (c) exposing each well of the multiwell plate to a test xenobiotic; (d) incubating the plate of c) to allow the yeast to grow; (e) collecting samples from d) and isolating DNA; and (f) detecting the relative abundance of the unique DNA barcodes; wherein a decrease in the abundance of a particular unique DNA barcode indicates that the xenobiotic metabolizing enzyme in the yeast strain containing that DNA barcode bioactivates the test xenobiotic and indicates that it is able to debilitate the target organism.

In an embodiment, the wells are tubes and the plate is a plate holding multiple tubes.

In some embodiments, different test xenobiotics will be in different wells of the multiwell plate as well as positive and negative controls.

In an embodiment, the method further comprises (g) pooling the yeast strains of the library of the one or more non-target organisms, (h) depositing the pooled library of g) into wells of a multiwell plate; (i) exposing each well of the multiwell plate to the test xenobiotic; (j) incubating the plate of i) to allow the yeast to grow; (k) collecting samples from j) and isolating DNA; and (l) detecting the relative abundance of the unique DNA barcodes; wherein the test xenobiotic is selective if it does not decrease the abundance of a particular DNA barcode of the yeast strains of the library of the one or more non-target organisms.

In other embodiments, the assay is used to test other compound-metabolizing enzymes.

Accordingly, the assay disclosed herein is used to identify substrates of xenobiotic metabolizing enzymes that are not bioactivated or to identify compounds that modify the enzymes. Accordingly, provided herein is an assay for identifying substrates of a xenobiotic metabolizing enzyme that are not bioactivated or compounds that modify xenobiotic metabolizing enzymes comprising (a) exposing a yeast expressing a heterologous xenobiotic metabolizing enzyme to a candidate substrate whilst simultaneously exposing the same yeast culture to a known xenobiotic that the enzyme metabolizes into a lethal metabolite; (b) exposing a second yeast culture of the same strain as in (a) to only the known xenobiotic that the enzyme metabolizes into a lethal metabolite; (c) comparing the viability of the yeast of (a) to the viability of yeast of (b) wherein an increase in the viability of the yeast of (a) indicates that the candidate substrate is a substrate or is a compound that decreases the activity of the xenobiotic metabolizing enzyme, optionally performing analytical techniques to ensure that the known xenobiotic that the enzyme metabolizes into a lethal metabolite has not decreased in abundance. In an embodiment, the known xenobiotic is the candidate xenobiotic identified in the assays disclosed herein.

In some embodiments, the assay is used to test P450 sequence variants. Accordingly, the assay described herein is modified to determine the effect of a variant of the sequence encoding the xenobiotic metabolizing enzyme. Accordingly also provided is an assay for determining whether a sequence variant of a xenobiotic metabolizing enzyme affects its functionality comprising (a) obtaining a yeast that heterologously expresses a variant of a xenobiotic metabolizing enzyme; (b) exposing the yeast of (a) to a known xenobiotic that the enzyme metabolizes into a lethal metabolite; (c) obtaining a yeast that heterologously expresses the non-variant version of a xenobiotic metabolizing enzyme described in (a); (d) exposing the yeast of (c) to a known xenobiotic that the enzyme metabolizes into a lethal metabolite; and (e) comparing the viability of the yeast of (b) to the yeast in (d), wherein an increase in the viability of the yeast in (b), indicates that the variant enzyme has decreased function. In an embodiment, the known xenobiotic is the candidate xenobiotic identified in the assays disclosed herein.

### Compounds and Methods and Uses Thereof

In yet another aspect, the present disclosure provides a family of compounds that incapacitate parasitic nematodes.

Accordingly, the present disclosure provides a method of treating or preventing a nematode infection comprising administering, to a subject in need thereof, an effective amount of a compound of Formula I, or a solvate thereof:

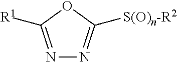

wherein:
R¹ is selected from phenyl and 6-membered heteroaryl, both of which are optionally substituted with one to three substituents independently selected from halo, NO₂, C₁₋₆alkyl, OC₁₋₆alkyl, C(O)C₁₋₆alkyl, OC(O)C₁₋₆alkyl and C(O)OC₁₋₆alkyl, wherein each alkyl group is optionally fluorosubstituted;
R² is selected from CF₃, C₁₋₆alkyl and C₁₋₆alkenyl; and
n is 0, 1 or 2.

In some embodiments, the present disclosure includes uses of pharmaceutical compositions comprising a compound of Formula (I) for treating or preventing a nematode infection or a disease, a disorder, or a condition arising from a nematode infection in a subject in need thereof.

In some embodiments, the present disclosure includes methods of treating or preventing a nematode infection comprising administering an effective amount of a nematicidal composition comprising a compound of Formula (I) described herein to a subject in need thereof.

In some embodiments, the present application includes uses of a nematicidal composition comprising a compound of Formula (I) described herein for treating or preventing a nematode infection or a disease, a disorder, or a condition arising from a nematode infection in a subject in need thereof.

In some embodiments, R¹ is phenyl optionally substituted with one to three substituents independently selected from Cl, Br, F, NO₂, C₁₋₄alkyl, OC₁₋₄alkyl, C(O)C₁₋₄alkyl, OC(O)C₁₋₄alkyl and C(O)OC₁₋₄alkyl, wherein each alkyl group is optionally fluorosubstituted.

In some embodiments, R¹ is phenyl optionally substituted with one to two substituents independently selected from Cl, Br, F, CH₃, CF₃, NO₂, CH₂CH₃, CH(CH₃)₂, C(CH₃)₃, OCH₃, OCF₃, OCH₂CH₃, OCH(CH₃)₂, OC(CH₃)₃, C(O)CH₃, C(O)CF₃, C(O)CH₂CH₃, C(O)CH(CH₃)₂, C(O)C(CH₃)₃, OC(O)CH₃, OC(O)CF₃, OC(O)CH₂CH₃, OC(O)CH(CH₃)₂, OC(O)C(CH₃)₃, C(O)OCH₃, C(O)OCF₃, C(O)OCH₂CH₃, C(O)OCH(CH₃)₂ and C(O)OC(CH₃)₃.

In some embodiments, R¹ is phenyl optionally substituted with one to two substituents independently selected from Cl, Br, F, NO₂, CH₃, CF₃, OCF₃, OCH₃, C(O)CH₃, OC(O)CH₃, and C(O)OCH₃.

In some embodiments, R¹ is phenyl optionally substituted with one to two substituents independently selected from Cl, Br, F, NO₂, CH₃ and C(O)OCH₃.

In some embodiments, R¹ is phenyl substituted with one to two substituents independently selected from Cl and Br.

In some embodiments, R¹ is a 6-membered heteroaryl optionally substituted with one to three substituents independently selected from Cl, Br, F, NO₂, C₁₋₄alkyl, OC₁₋₄alkyl, C(O)C₁₋₄alkyl, OC(O)C₁₋₄alkyl and C(O)OC₁₋₄alkyl.

In some embodiments, R¹ is a 6-membered heteroaryl optionally substituted with one to two substituents independently selected from Cl, Br, F, NO₂, CH₃ and C(O)OCH₃.

In some embodiments, R¹ is an unsubstituted 6-membered heteroaryl.

In some embodiments, the 6-membered heteroaryl is selected from pyridinyl, pyrimidinyl and pyrazinyl.

In some embodiments, the 6-membered heteroaryl is pyridinyl.

In some embodiments, R² is selected from C₁₋₄alkyl and C₁₋₄alkenyl.

In some embodiments, R² is selected from CH₃, CH₂CH₃, CH₂CH₂CH₃ and CH₂CH₂CH₂CH₃.

In some embodiments, R² is selected from CH═CH₂, CH₂C═CH₂, CH₂CH₂C═CH₂ and CH₂CH═CHCH₃.

In some embodiments, R² is CH₂C═CH₂.

In some embodiments, R² is CF₃.

In some embodiments, n is 0.

In some embodiments, the compound of Formula I is selected from:

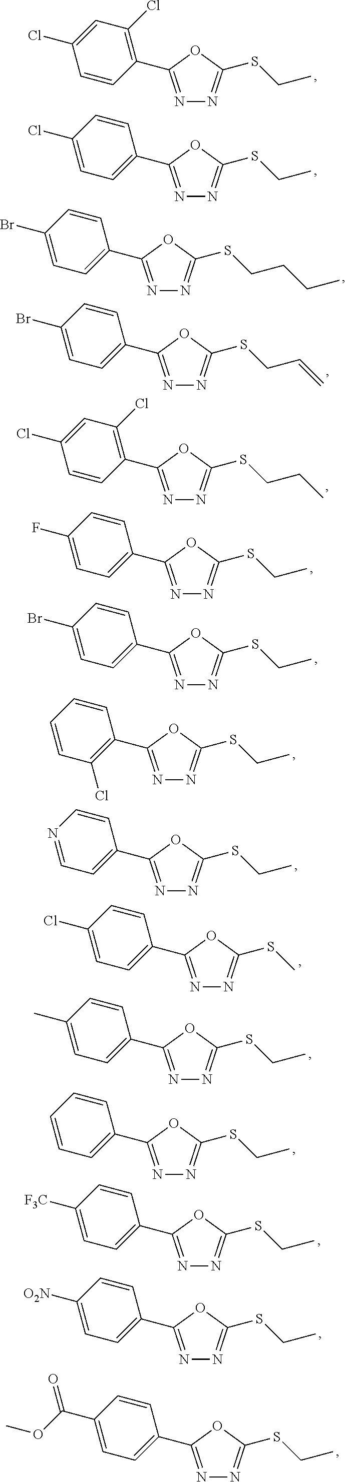

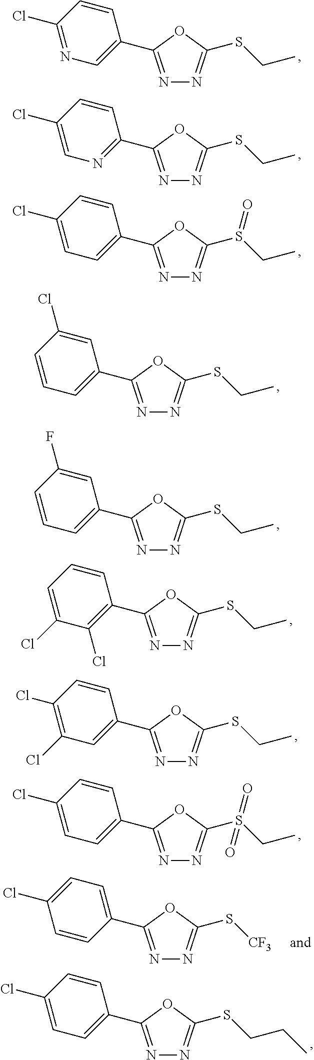

or a solvate thereof.

In some embodiments, the infection is an infection of a nematode of a species selected from Cooperia oncophora, and Haemonchus contortus.

In some embodiments, the infection is an infection of a nematode of a species selected from members of the genuses Pratylenchus, Heterodera, Meloidogyne, Ditylenchus or Pratylenchus. In some embodiments the nematodes are of the species Heterodera glycines, Globodera pallida, Meloidogyne javanica, Meloidogyne incognita, Meloidogyne arenaria, Radopholus similis, Longidorus elongatus, Meloidogyne hapla, Ditylenchus dipsaci or Pratylenchus penetrans.

Treatment methods comprise administering to a subject one or more compounds of the application, and optionally consists of a single administration, or alternatively comprises a series of administrations. The length of the treatment period depends on a variety of factors, such as the severity of the infection, disease, disorder or condition, the age of the subject, the dosage of the one or more compounds of the application, the activity of one or more compounds of the application, or a combination thereof.

In an embodiment, the one or more compounds of the application are administered or used as soon as possible after exposure to the nematode. In an embodiment, the one or more compounds of the application are administered or used until treatment of the nematode infection, disease disorder or condition is achieved. For example, until complete elimination of the nematode is achieved, or until the number of nematode has been reduced to the point where the subject's defenses are no longer overwhelmed and can kill any remaining nematode.

In an embodiment, the methods of the present disclosure comprise administering an effective amount of a compound or a composition of the application to a subject selected from humans, mammals, birds, vertebrates, plants, seeds, and soil.

In an embodiment, the uses of the present disclosure of a compound or a composition of the disclosure are in a subject selected from humans, mammals, birds, vertebrates, plants, seeds, and soil.

In some embodiments, the nematode infects plants and the nematicidal composition is administered to the soil or to plants. In some embodiments, the nematicidal composition is administered to soil before planting. In some embodiments, the nematicidal composition is administered to soil after planting. In some embodiments, the nematicidal composition is administered to soil using a drip system. In some embodiments, the nematicidal composition is administered to soil using a drench system. In some embodiments, the nematicidal composition is administered to plant roots or plant foliage (e.g., leaves, stems). In some embodiments the nematicide composition is tilled into the soil or administered in furrow. In some embodiments, the nematicidal composition is administered to seeds.

In some embodiments, the nematode parasite infects a vertebrate. In some embodiments, the nematicidal composition is administered to non-human vertebrate. In some embodiments, the nematicidal composition is administered to a human. In some embodiments, the nematicidal composition is formulated as a drench to be administered to a non-human animal. In some embodiments, the nematicidal composition is formulated as an orally administered drug. In some embodiments, the nematicidal composition is formulated as an injectable drug. In some embodiments, the nematicidal composition is formulated for topical applications such as pour-ons, or for the use in tags or collars.

In some embodiments, the methods of the disclosure comprise administering a compound or a composition of the application through one or more means selected from pre-planting, post-planting, as a feed additive, a drench, an external application, a pill and by injection.

In some embodiments, the present disclosure includes methods of reducing the viability or fecundity or slowing the growth or development or inhibiting the infectivity of a nematode using a compound or a composition of the disclosure as described herein.

In some embodiments, the present disclosure includes methods of reducing the viability or fecundity or slowing the growth or development or inhibiting the infectivity of a nematode using a compound or a composition of the disclosure as described herein, the methods comprising administering a compound or a composition of the disclosure to subject selected from a human, a mammal, a bird, a vertebrate in general, a plant, a seed, or soil. In some examples, the bird can be a domesticated fowl; the mammal can be a domesticated animal and/or livestock.

The dosage of the one or more compounds of the disclosure, varies depending on many factors such as the pharmacodynamic properties thereof, the mode of administration, the age, health and weight/mass of the subject, the nature and extent of the symptoms, the frequency of the treatment and the type of concurrent treatment, if any, and the clearance rate in the subject to be treated. One of skill in the art can determine the appropriate dosage based on the above factors. The one or more compounds of the disclosure may be administered initially in a suitable dosage that may be adjusted as required, depending on the response.

Compounds can be tested for anthelmintic activity using methods known in the art. For example, the compound is combined with nematodes, e.g., in a well of microtiter dish, in liquid or solid media or in the soil containing the agent. Staged nematodes are placed on the media. The time of survival, viability of offspring, and/or the movement of the nematodes are measured. An agent with “anthelmintic or anthelminthic or antihelmthic activity” can, for example, reduce the survival time of adult nematodes relative to unexposed similarly staged adults, e.g., by about 20%, 40%, 60%, 80%, or more. In the alternative, an agent with “anthelmintic or anthelminthic or antihelminthic activity” may also cause the nematodes to cease replicating, regenerating, and/or producing viable progeny, e.g., by about 20%, 40%, 60%, 80%, or more. The effect may be apparent immediately or in successive generations.

The compounds of Formula I are commercially available or can be prepared by various synthetic processes known in the art. The choice of particular structural features and/or substituents may influence the selection of one process over another. The selection of a particular process to prepare a given compound of Formula I is within the purview of the person of skill in the art. Some starting materials for preparing compounds of the present application are available from commercial chemical sources. Other starting materials, for example as described below, are readily prepared from available precursors using straightforward transformations that are well known in the art.

The compounds of Formula I generally can be prepared according to the processes illustrated in the Schemes below. In the structural formulae shown below the variables are as defined in Formula I unless otherwise stated. A person skilled in the art would appreciate that many of the reactions depicted in the Schemes below would be sensitive to oxygen and water and would know to perform the reaction under an anhydrous, inert atmosphere if needed. Reaction temperatures and times are presented for illustrative purposes only and may be varied to optimize yield as would be understood by a person skilled in the art.

In some embodiments, compounds of Formula I are prepared as shown in Scheme 1:

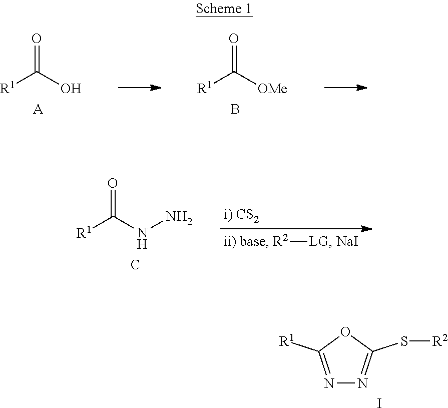

Therefore, in some embodiments, a carboxylic acid of Formula A, wherein R¹ is as defined in Formula I, is first converted to the corresponding alkyl ester, such as a methyl ester of Formula B, which is then converted to the corresponding hydrazide of Formula C, for example by reaction with hydrazine in a suitable alcohol solvent (e.g. methanol). The hydrazide of Formula C is then transformed into a compound of Formula I wherein n is 0, for example, using a two-step reaction comprising a first reaction with carbon disulfide in a suitable solvent such as dimethyl formamide (DMF) followed by reaction with a compound of the formula R²-LG, wherein R² is as defined in Formula I and LG is a suitable leaving group, such as halo (e.g. Br), in the presence of a non-nucleophilic base such as an organic amine base (e.g. triethylamine) and sodium iodide.

An alternate method to the compounds of Formula I is shown in Scheme 2:

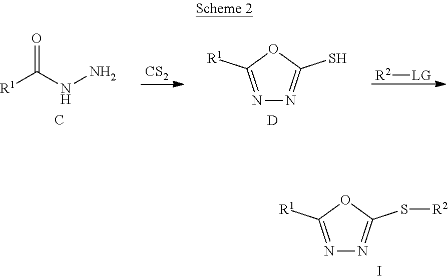

Therefore, in some embodiments, compounds of Formula C are reacted with carbon disulfide in the presence of an inorganic base in an aqueous solvent to provide the thiol of Formula D which is then reacted with a compound of the formula R²-LG, wherein R² is as defined in Formula I and LG is a suitable leaving group, such as halo (e.g. Br), in the presence of a non-nucleophilic base to provide the compounds of Formula I.

Compounds of Formula I wherein n is 1 or 2 are available from compounds of Formula I wherein n is 0 using known sulfur oxidation conditions. For example, to obtain compounds of Formula I wherein n is 1, a compound of Formula I wherein n is 0 is reacted with a suitable oxidizing agent such as m-chloroperbenzoic acid (m-CPBA) in a suitable organic solvent. Further, to obtain compounds of Formula I wherein n is 2, a compound of Formula I wherein n is 0 is for example reacted with a suitable oxidizing agent such as potassium permanganate (KMnO₄) in a suitable polar organic solvent.

The formation of solvates will vary depending on the compound and the solvate. In general, solvates are formed by dissolving a compound in the appropriate solvent and isolating the solvate by cooling or using an antisolvent. The solvate is typically dried or azeotroped under ambient conditions. The selection of suitable conditions to form a particular solvate can be made by a person skilled in the art. Examples of suitable solvents are ethanol, water and the like. When water is the solvent, the molecule is referred to as a “hydrate”.

Throughout the processes described herein it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include, for example, straight and reversed phase chromatography on column or rotating plate, recrystallisation, distillation and liquid-liquid or solid-liquid extraction, which will be readily understood by one skilled in the art.

The products of the processes of the application may be isolated according to known methods, for example, the compounds may be isolated by evaporation of the solvent, by filtration, centrifugation, chromatography or other suitable method.

One skilled in the art will recognize that where a reaction step of the present application is carried out in a variety of solvents or solvent systems, said reaction step may also be carried out in a mixture of the suitable solvents or solvent systems.

Certain compounds of Formula I are novel, therefore the present application also includes the novel compounds of Formula I or a solvate thereof. In some embodiments, the present application includes a compound selected from:

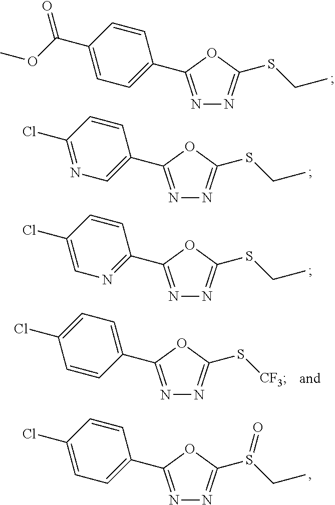

or a solvate thereof.

The above disclosure generally describes the present application. A more complete understanding can be obtained by reference to the following specific examples. These examples are described solely for the purpose of illustration and are not intended to limit the scope of the application. Changes in form and substitution of equivalents are contemplated as circumstances might suggest or render expedient. Although specific terms have been employed herein, such terms are intended in a descriptive sense and not for purposes of limitation.

### EXAMPLES

The following non-limiting examples are illustrative of the present disclosure:

### Example 1: The CYPce Assay

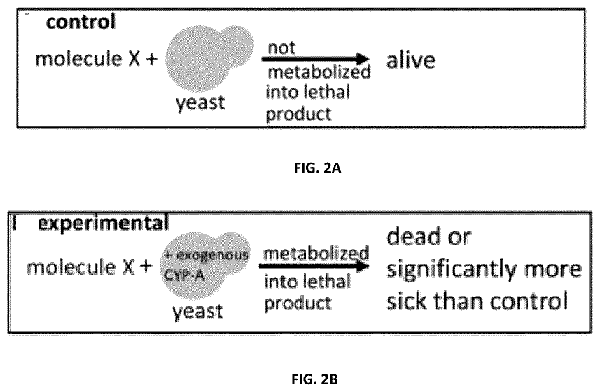

CYPce (pronounced sip-sea) is an acronym for the heterologous expression of CYPs in S. cerevisiae for the purpose of identifying P450s that can bioactivate small molecules into lethal products that may or may not be reactive. The CYPce assay relies on culturing a single strain of yeast (S. cerevisiae) (or any convenient cell type in other embodiments) that expresses a single P450 enzyme (or any other small molecule modifying enzyme in other embodiments) from a single targeted organism (FIG. 2).

With that single strain, a library of small molecules are screened for those that can be bioconverted by the heterologously-expressed enzyme into a product that kills the cells expressing the enzyme. As set out herein, the growth of cells was measured, for example, by a variety of means, including optical density. Hits were determined by comparing the growth rate of the yeast expressing the P450 from the targeted organism in the presence of the test molecule to a control, which did not express transgenic exogenous P450s. Experimental samples that grew significantly worse than the controls were considered hits.

Additional CYPce experiments were run in parallel to those focused on strains expressing P450s from targeted organisms to identify small molecule-P450 pairs that can be exploited to selectively kill the target organism. This was done by performing screens with the same set of molecules used with the P450s from targeted organisms, but instead with yeast strains expressing P450s from non-targeted organisms. In this way, small molecule hits that were selectively bioactivated by P450s from targeted organism and not relevant non-targeted organisms were identified.

Hits were then tested against the respective targeted organism. To date, CYPce has been used to demonstrate that nematode P450s bioactivate selectivin (6-(4-chlorophenyl)imidazo[2,1-b]thiazole)) and cyproside-3 (2-(4-chlorophenyl)-5-(ethylthio)-1,3,4-oxadiazole) into lethal reactive electrophile products (see examples 2-5, below). CYPce has also been used to successfully identify small molecules that can be selectively bioactivated by mosquito P450s (see example 6 below).

CYPce was used to reveal small molecules that are metabolically bioactivated into lethal products by enzymes, including Cytochrome P450s: Through CYPce technology, molecules that may selectively debilitate target organisms of interest were readily identified. Follow up in vitro or in vivo assays with the pathogen, parasite or pest of interest (and respective relevant non-targeted systems) confirmed the translation of hits to the targeted organism.

CYPce is also used to reveal lethal metabolites that are not reactive, which they themselves will be useful cidal agents. Metabolites that are generated by enzymes/P450s that kill cells are revealed by CYPce and downstream analyses. These are purified and have utility on their own.

### Example 2

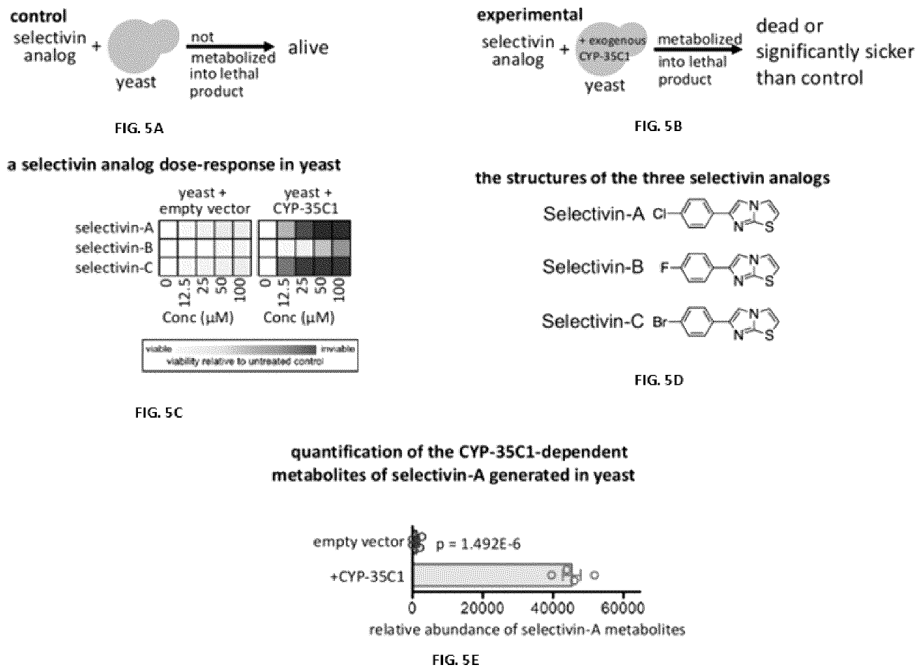

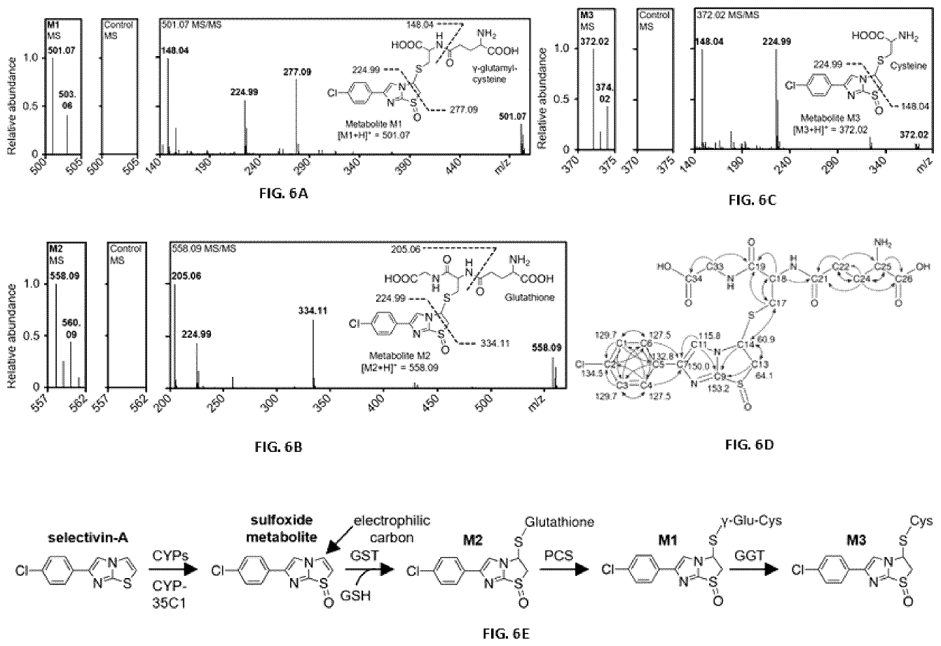

The CYPce assay was used to show that selectivin structural analogs, including selectivin-A (6-(4-chlorophenyl)imidazo[2,1-b]thiazole), kills yeast in a manner dependent on the C. elegans P450 CYP-35C1 (FIG. 5A-5D). Analytical chemistry (HPLC) shows that selectivin-A is metabolized by CYP-35C1 in yeast (FIG. 5E). Analytical chemistry (LCMS and NMR) shows how selectivin-A is metabolized by CYP-35C1 into a reactive lethal product (FIG. 6).

### Example 3

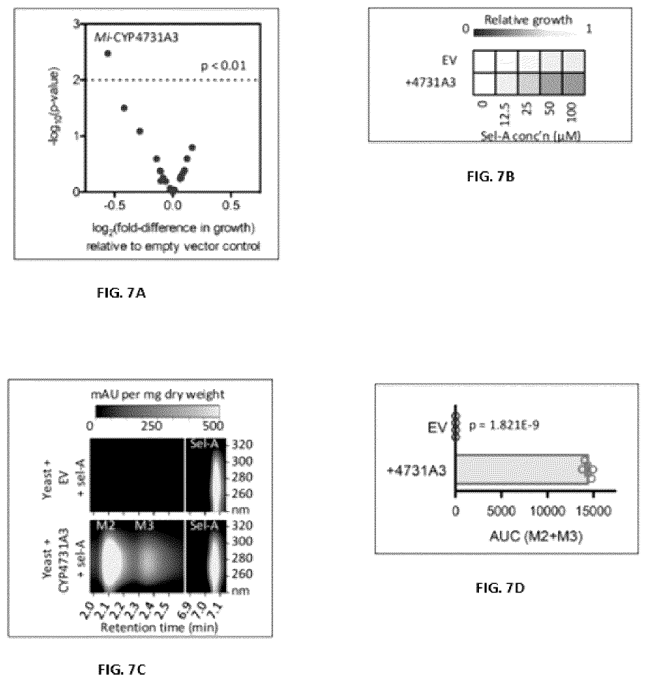

The CYPce assay was used in a screen to reveal which P450 enzyme from the plant parasitic nematode Meloidogyne incognita (Mi) kills yeast in a selectivin-A (6-(4-chlorophenyl)imidazo[2,1-b]thiazole)-dependent manner (FIG. 7A). This screen revealed that selectivin-A kills yeast in a Mi-CYP4731A3-dependent manner (FIGS. 7A and 7B). Analytical chemistry (HPLC) showed that selectivin-A was metabolized by Mi-CYP4731A3 in yeast (FIGS. 7C and 7D).

### Example 4

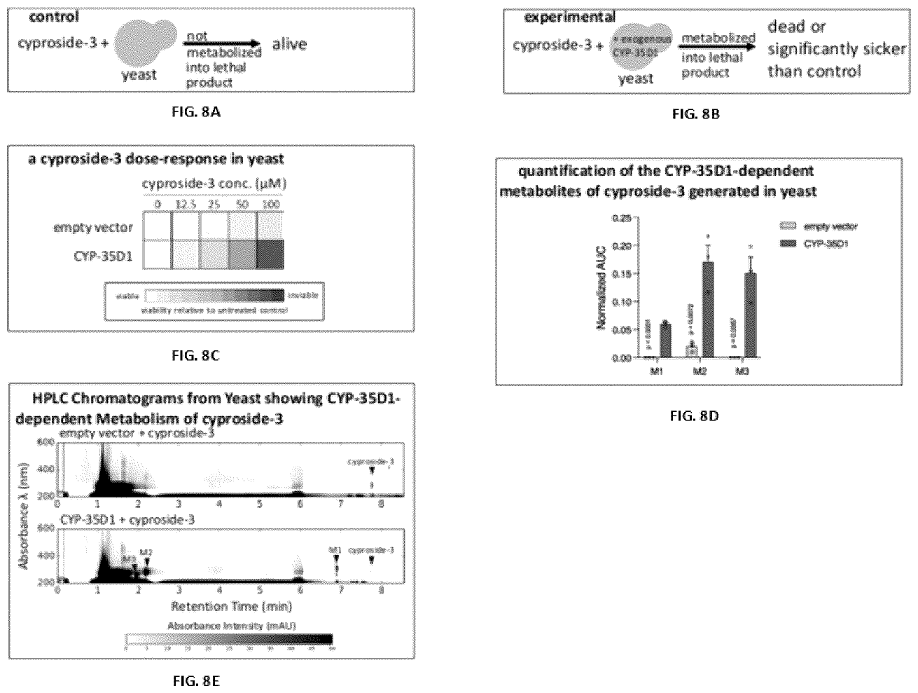

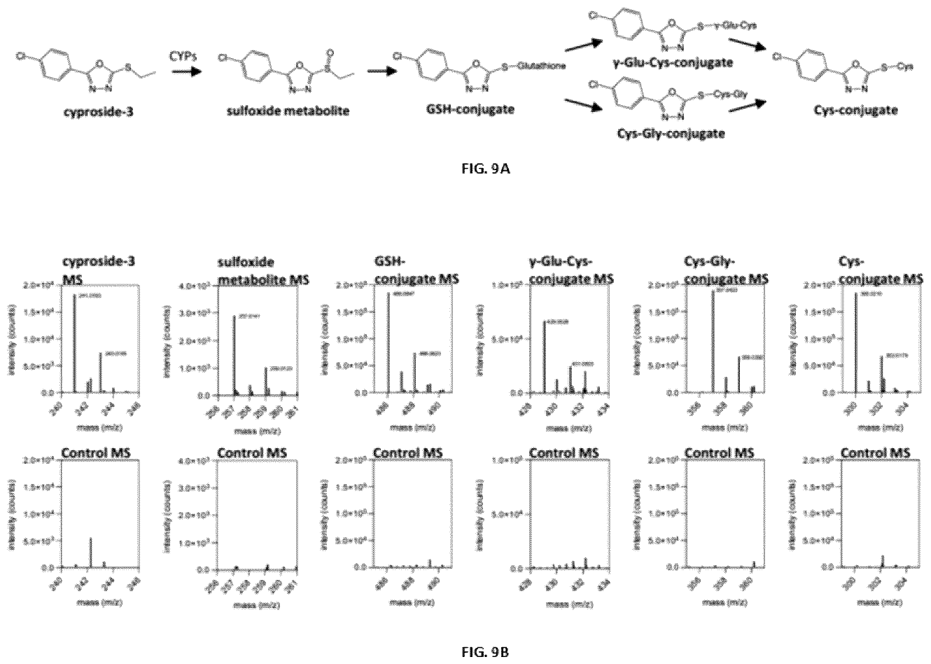

The CYPce assay showed that cyproside-3 ((2-(4-chlorophenyl)-5-(ethylthio)-1,3,4-oxadiazole)) killed yeast in a manner dependent on C. elegans P450 CYP-35D1 (FIG. 8A-8C). Analytical chemistry (HPLC) showed that cyproside-3 was metabolized by CYP-35D1 into a reactive product (FIGS. 8D and 8E). Analytical chemistry (LCMS) showed how cyproside-3 is metabolized by CYP-35D1 into a reactive product (FIG. 9).

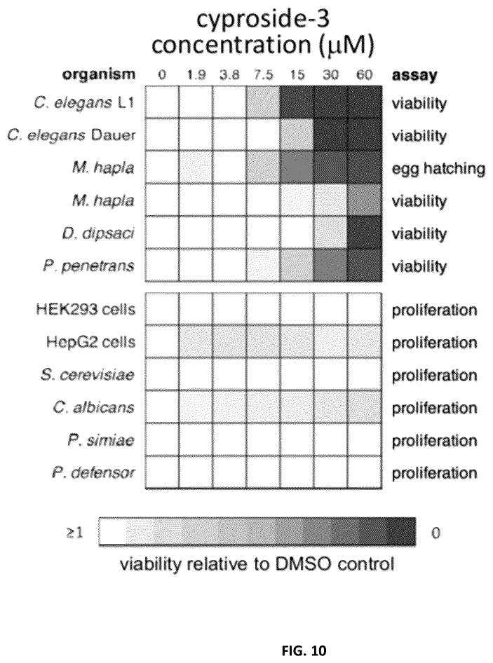

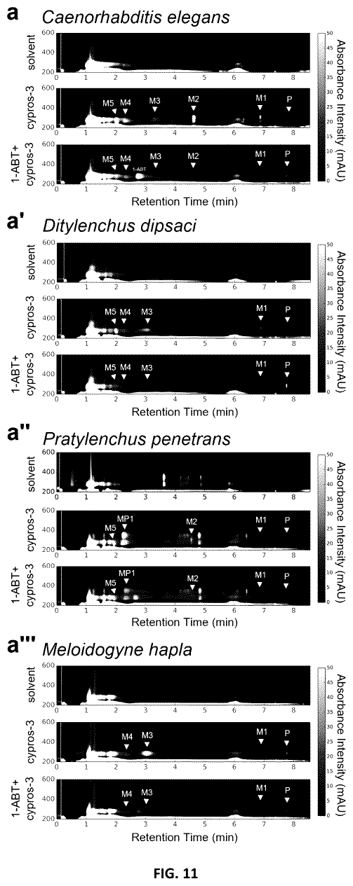

The utility of cyproside-3 was evident from its phylogenetic activity profile, showing high selectivity for all nematode species tested (including non-parasitic and plant parasitic species), but little-to-no activity against non-targeted cells and organisms (FIG. 10).

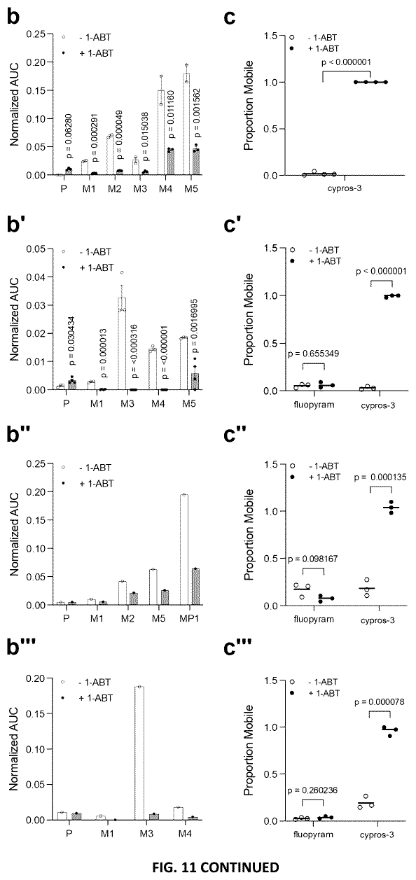

Using the pan-P450 inhibitor (1-ABT), cyproside-3's metabolism was inhibited in vivo (FIGS. 11A and 11B). 1-ABT also inhibited cyproside-3's effects on the worms (as measured by mobility) (FIG. 11C). Together, these data show that cyproside-3 was being bioactivated in whole worms into lethal products by P450s.

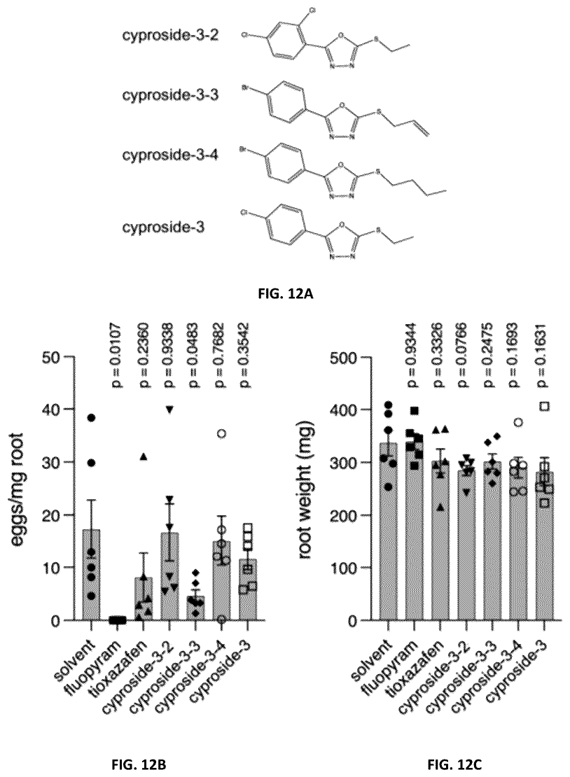

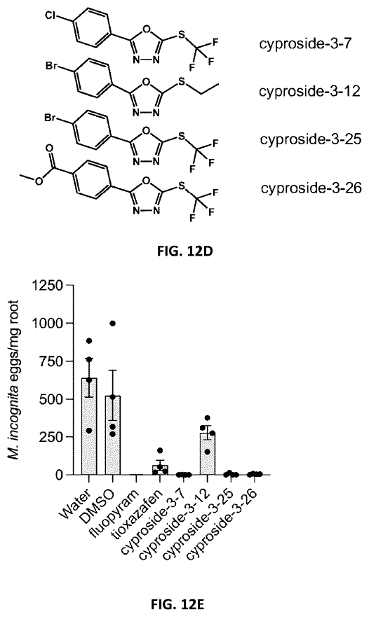

A small survey of cyproside-3 and structural analogs demonstrated activity againstthe plant parasitic nematode Meloidogyne incognita in soil based-assays (FIGS. 12A, 12B, 12D and 12E). There were no significant adverse effects on plant growth by the cyproside-3s as evident by the resulting root mass (FIG. 12C).

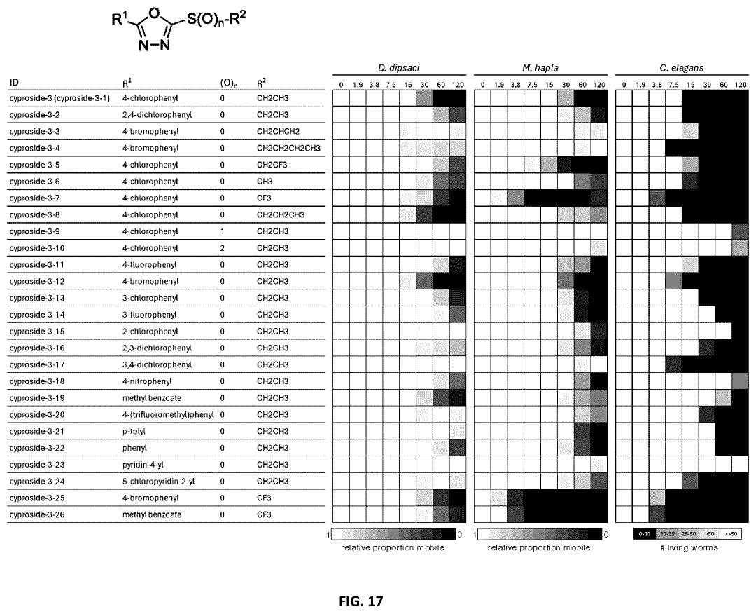

FIG. 17 shows the cyproside-3 structural analogs that have either been tested for activity against nematodes or will be tested against nematodes.

### Example 5

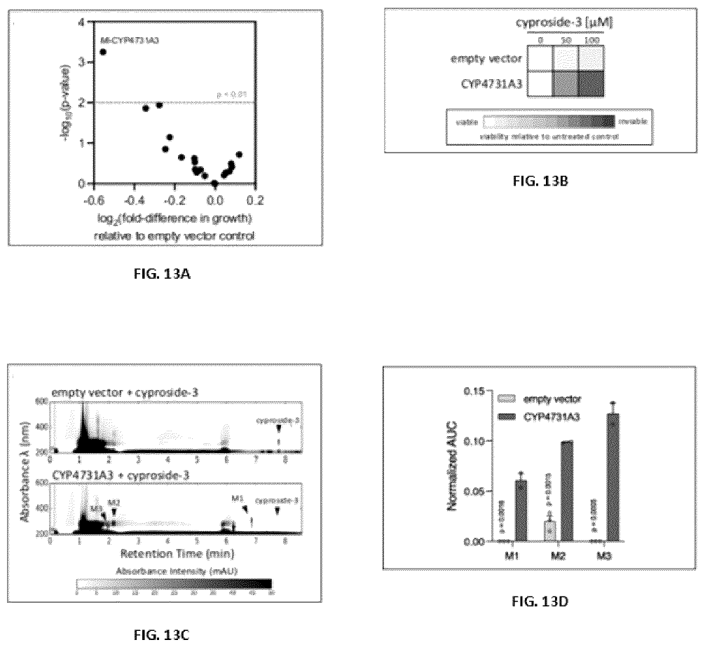

The CYPce assay was used in a screen to identify which P450 enzyme from the plant parasitic nematode Meloidogyne incognita (Mi) killed yeast in a cyproside-3 ((2-(4-chlorophenyl)-5-(ethylthio)-1,3,4-oxadiazole))-dependent manner (FIG. 13A). This screen revealed that cyproside-3 can kill yeast in a Mi-CYP4731A3-dependent manner (FIGS. 13A and 13B). Analytical chemistry (HPLC) showed that cyproside-3 is metabolized by Mi-CYP4731A3 in yeast (FIGS. 13C and 13D).

### Example 6

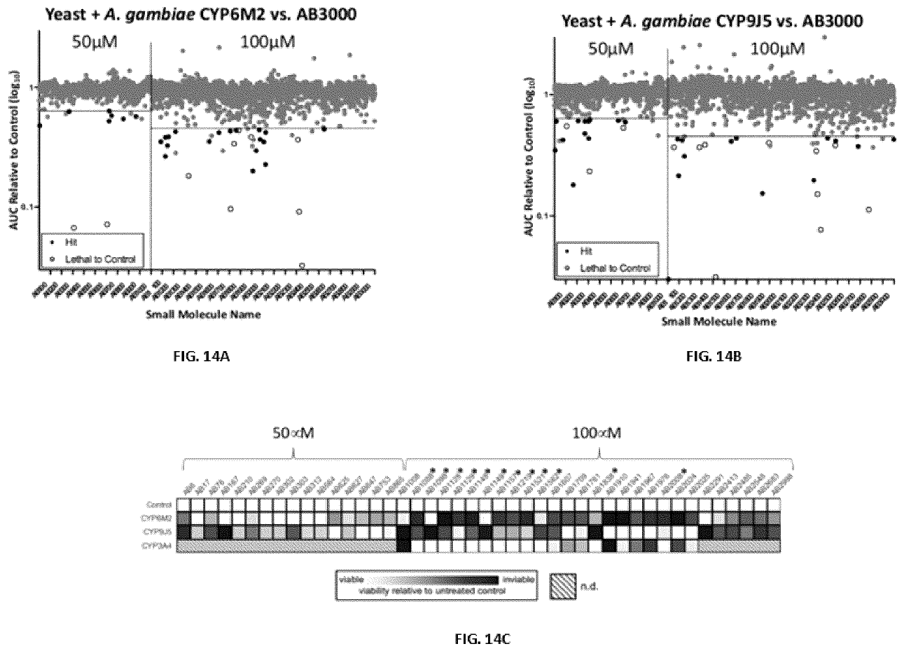

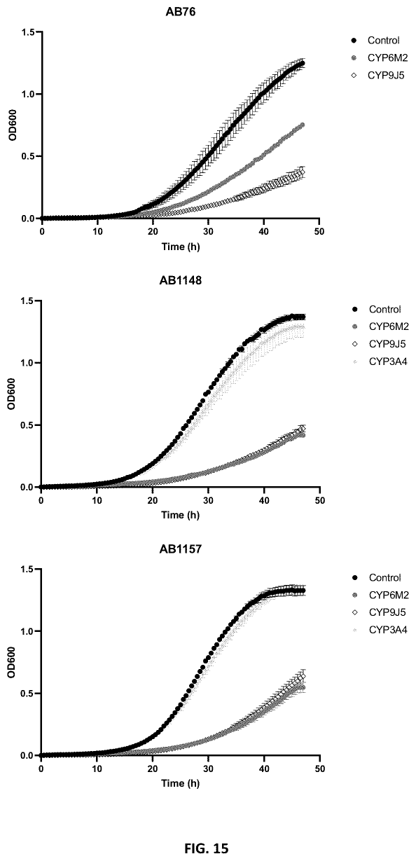

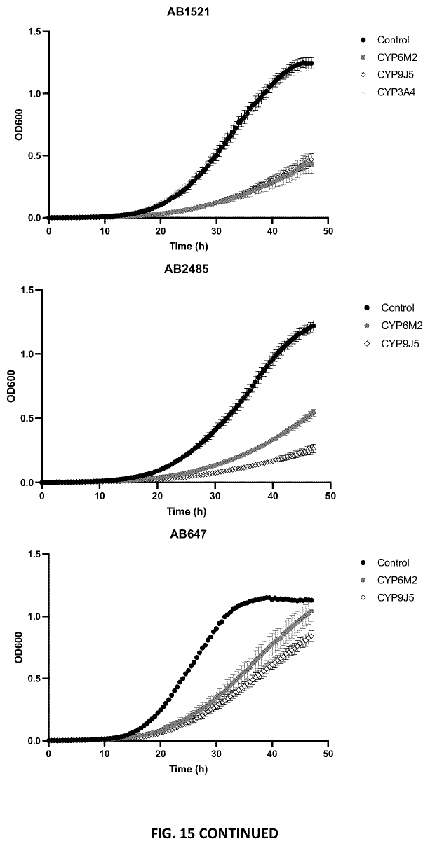

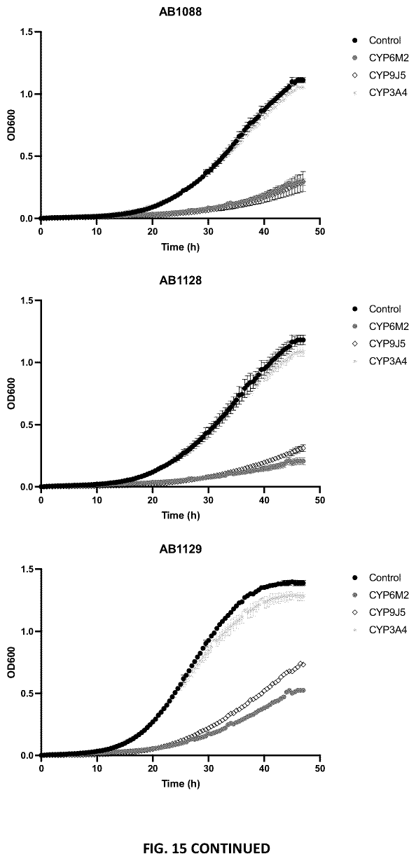

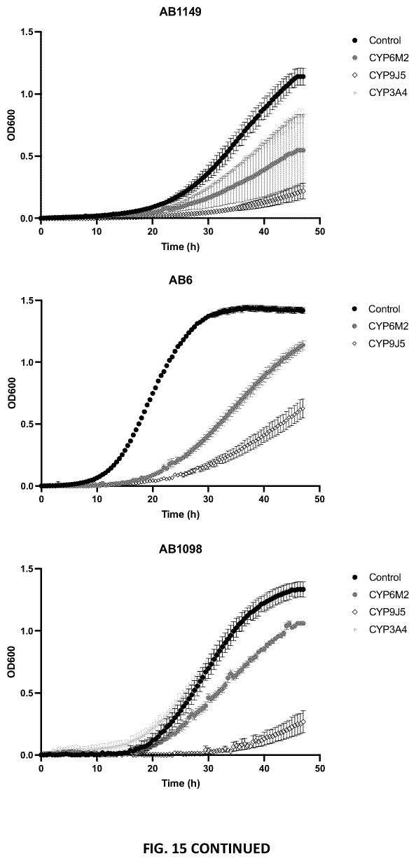

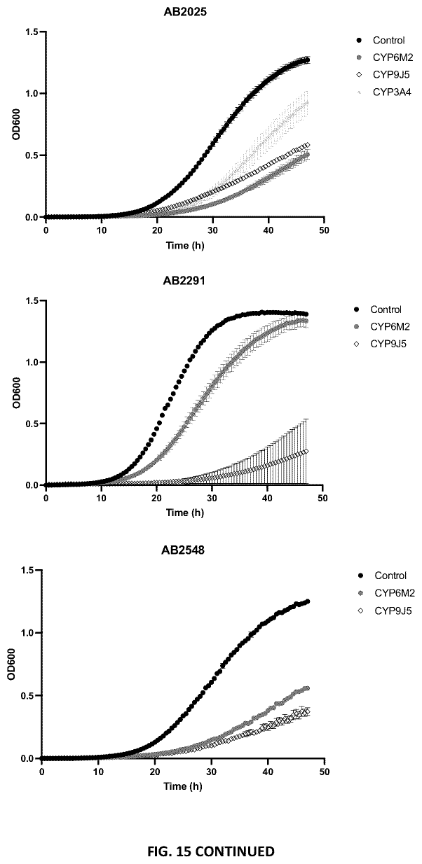

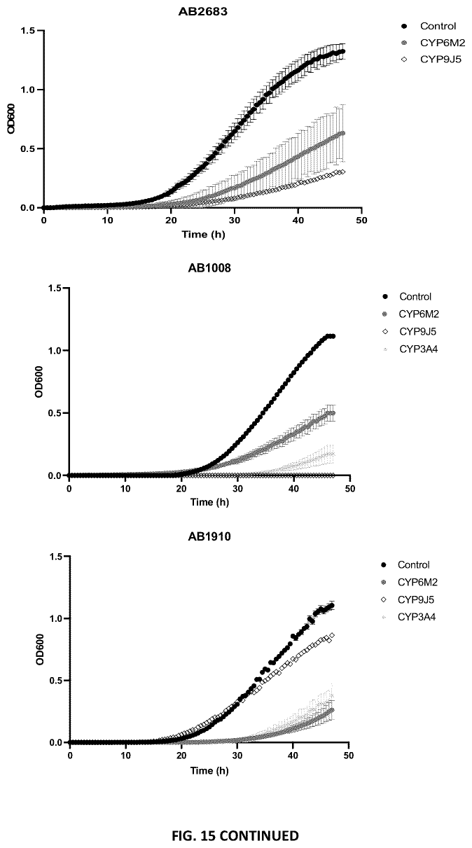

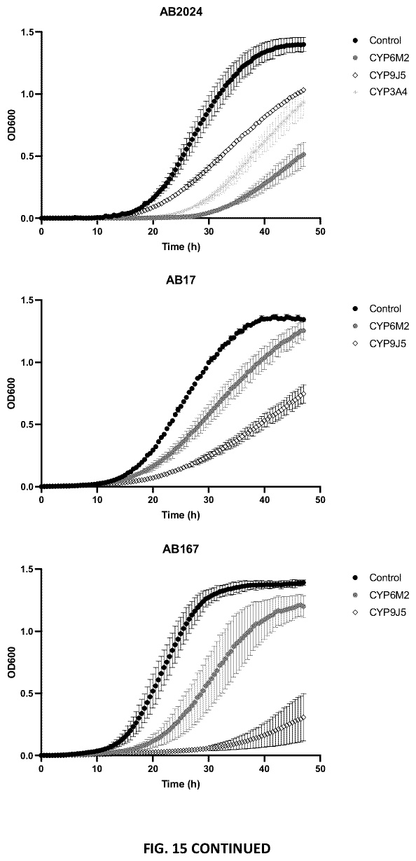

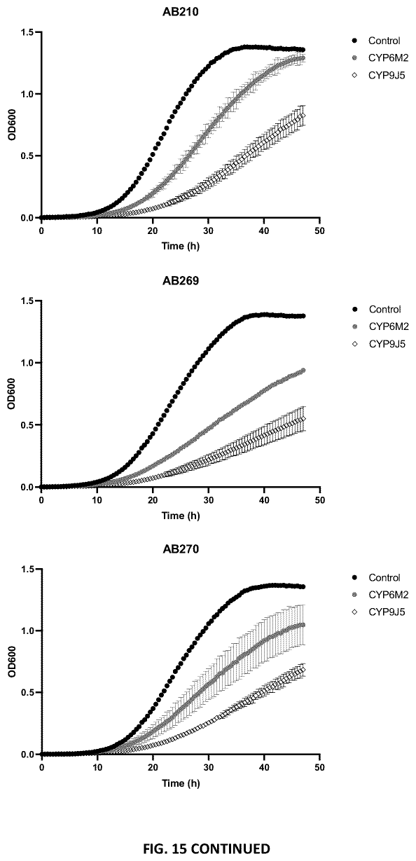

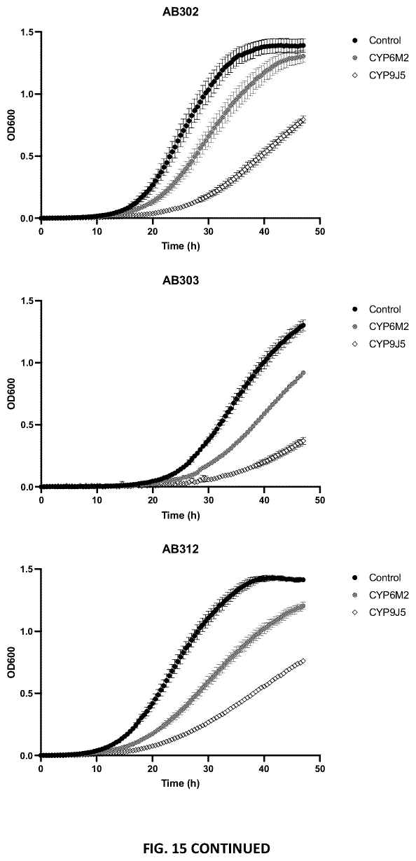

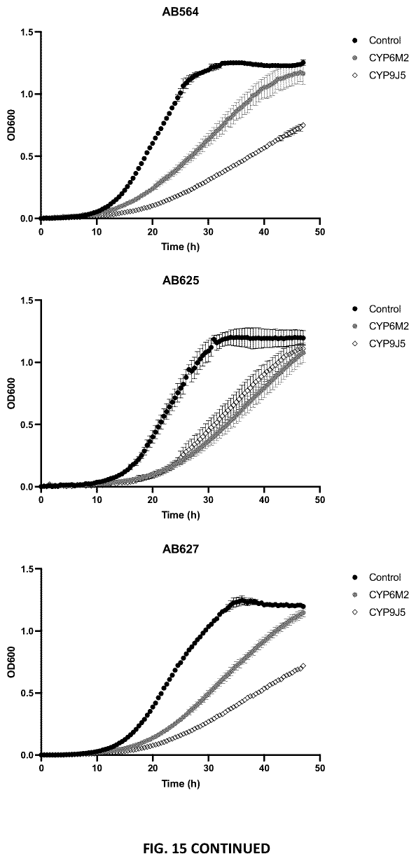

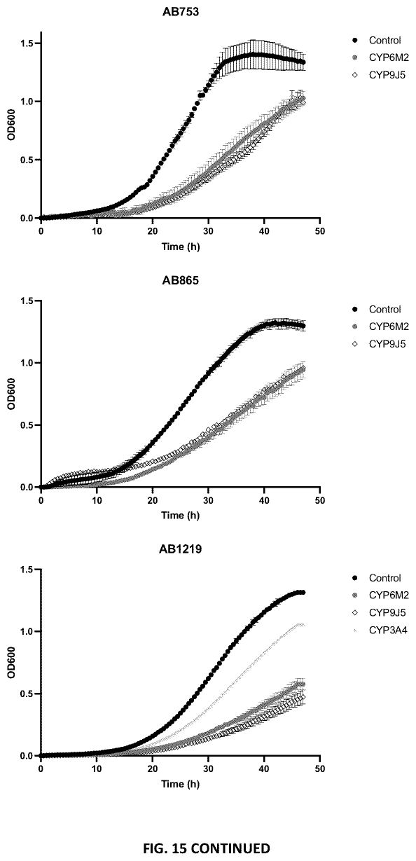

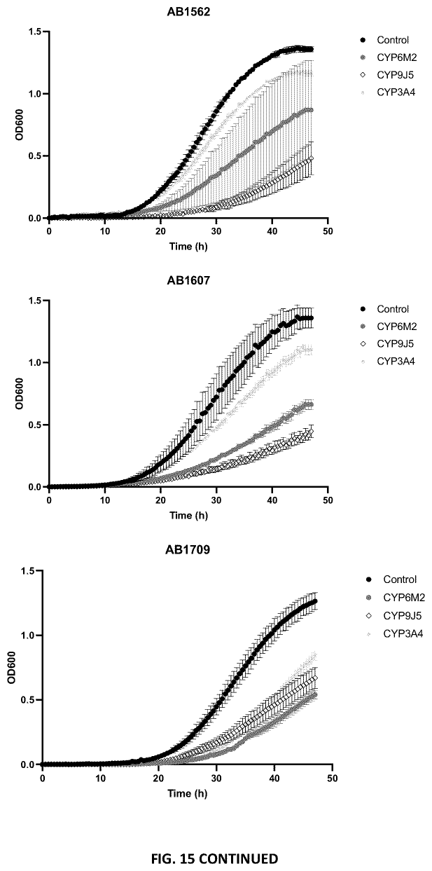

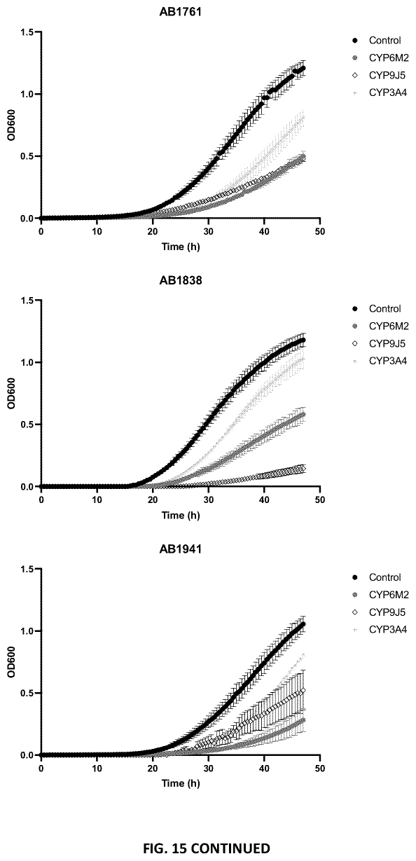

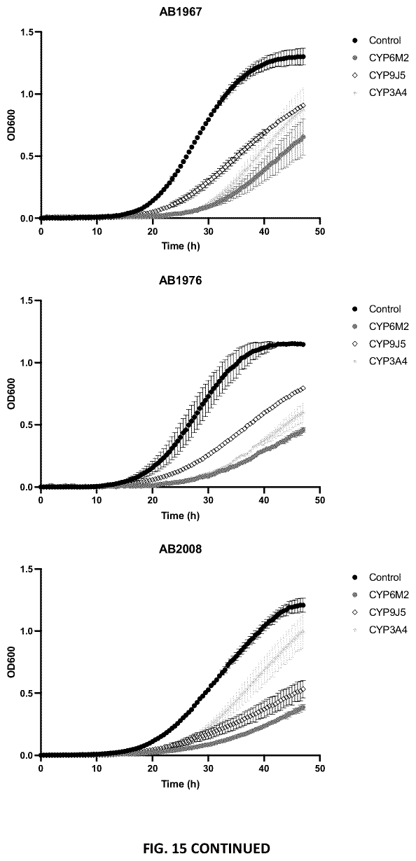

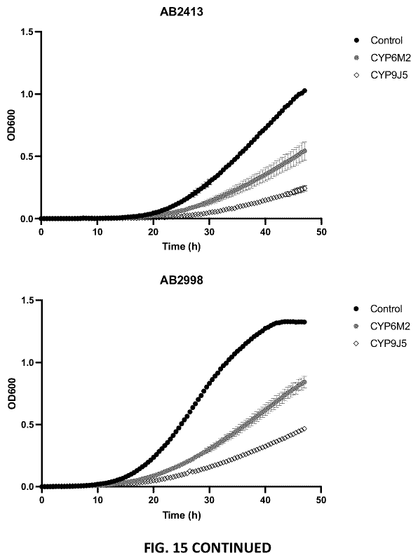

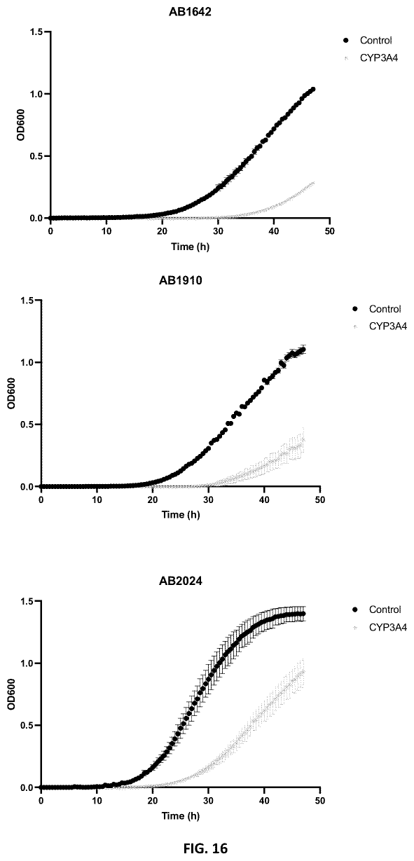

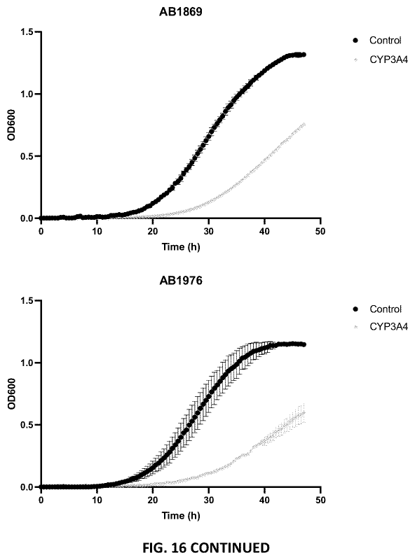

The CYPce assay was used in a screen to identify small molecules that can kill yeast in a mosquito P450 (CYP6M2 and CYP9J5)-dependent manner (FIGS. 14 and 15). Some of the resulting hits were also tested against human CYP3A4, revealing selective activity against the two mosquito CYPs (FIG. 14C). The activity of the hits are shown (FIG. 15).

### Example 7: The PEXIL Assay

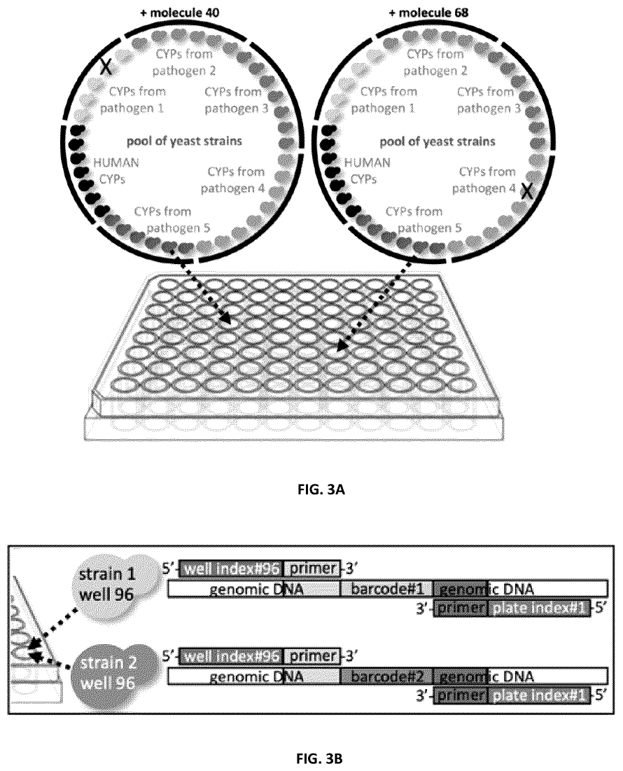

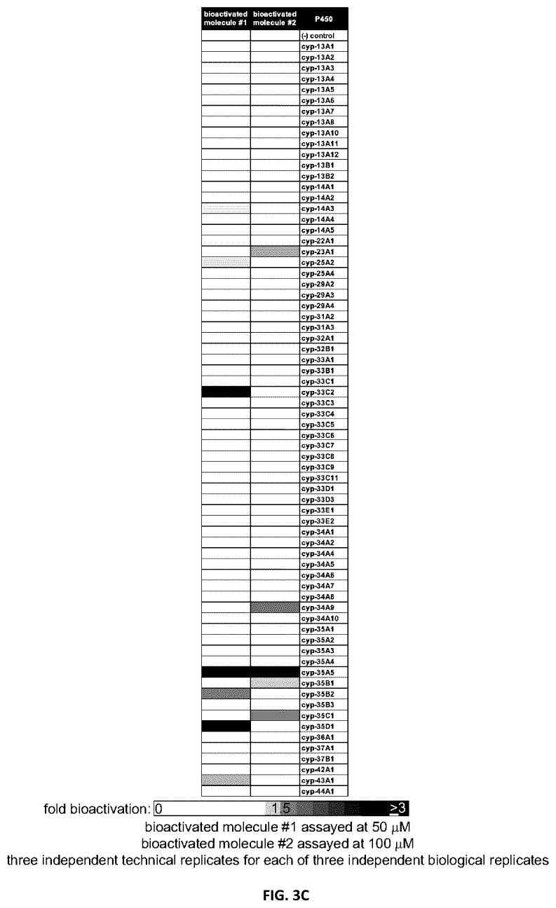

PEXIL is an acronym for massively paralleled screens for enzyme-activated xenobiotic-induced lethality. PEXIL is an extension of CYPce whereby a collection of multiple yeast strains (or any convenient cell type in other embodiments), each of which express a different P450 enzyme (or any other small molecule modifying enzyme in other embodiments) is assembled (FIG. 3). Each of the different yeast strains are uniquely marked with a DNA barcode (a unique string of DNA bases) that are flanked by common PCR primer binding sites that are common to all the strains' DNA barcodes. The multiple strains are combined into a pool, which is then tested against a library of small molecules systematically in multiwell plate format. The same pool is used in each of the wells, but a different small molecule is added to each of the wells. To identify which strain within the pool, if any, is bioconverting the small molecule into a lethal product, a DNA preparation is made from each well and the barcodes are amplified by PCR and sequenced. Strains that are sick or dead within the pool will have less barcode sequenced from the pool. In this way, which P450s within the pool can metabolize which small molecules into lethal products in a massively paralleled manner are identified.

Below, the methodology is described in a stepwise manner.

### PEXIL Step 1. Assemble a Library of CYP-Expressing Yeast Strains.

The coding sequence of known or suspected xenobiotic-metabolizing enzymes from one or more target organisms are individually cloned into a vector that allows for controlled expression in yeast., Codons may be optimized for expression in the heterologous cell being used. The coding sequence can be tagged with other proteins or peptides to enable tracking of the enzyme's expression level and sub-cellular localization. Unique DNA barcodes can be inserted on every unique resulting plasmid (Binan et al., 2019; McMahon et al., 2011). Alternatively, DNA barcodes can be inserted into the genome of each unique yeast strain that expresses a unique candidate xenobiotic-metabolizing enzyme. The unique DNA barcodes allows the tracking of the relative abundance of each yeast strain within a mix population of yeast strains using next-generation sequencing technologies. The vector that drives the expression of the heterologous enzyme can be maintained as a plasmid or can be integrated into either a fixed or random location within the yeast genome. The library of yeast strains that heterologously express xenobiotic-metabolizing enzymes from the targeted organisms is called the ‘targeted library’.

In addition, a sub-library of xenobiotic-metabolizing enzymes from non-target organisms can also be individually cloned into a vector that allows for controlled expression in yeast similar to above. Depending on the end goals of the pipeline, non-target organism may include humans, pets, livestock, other mammals, pollinators, birds, fish, plants etc. The library of yeast strains that heterologously express xenobiotic-metabolizing enzymes from the non-targeted organisms is called the ‘counter-screen or non-target library’.

The xenobiotic-metabolizing enzymes may require co-factors to facilitate optimal activity. In the case of heterologously-expressed P450s, the co-factor POR (P450 oxidoreductase) and/or cytochrome b5 from the same organism from which the P450 was derived can also be cloned into the respective yeast strains. Co-factors can be expressed from the same transgene that expresses the xenobiotic-metabolizing enzyme or from a different transgene than that which expresses the xenobiotic-metabolizing enzyme. Co-factors can be integrated into the genome of the respective yeast strain that also expresses the heterologous xenobiotic-metabolizing enzyme. Co-factors may be unnecessary for the activity of the P450.

PEXIL Step 2. Screen the Strains within the Targeted and Counter-Screen Libraries for Selective Sensitivities to Small Molecules.

In many cases, screens in tractable single cell models like yeast is of much higher throughput compared to screening directly against the individual pathogen, parasite or pest of interest.

Set-Up: Targeted libraries of yeast may or may not be screened together with the counter-screen libraries of yeast. The barcoded yeast strains are pooled together before the screen (FIG. 3). Aliquots of the pooled library are deposited into the wells of multiwell plates. The pooling can be limited to a single tube or well. Into each well, xenobiotics are deposited at a desired concentration. Singlet, duplicate, triplicate etc repeats of the test are done. Solvent-only controls (for example, DMSO solvent), and positive pro-xenobiotic controls (for example, xenobiotic-CYP positive controls), are added when available and as desired. The plates are incubated at the desired growth temperature for the cell type being used. At the assay endpoint, an aliquot of the pooled library from each well are moved into a fresh plate. Samples from intermediate time points might also be collected into a new fresh plate. Individual DNA preps of the collected samples are made. The DNA barcodes from each well are amplified by PCR. The PCR primers used to amplify the DNA barcodes from each well may also have barcodes that are specific to individual plates. In this way, PCR products from all wells and all plates are combined into a single sample and the relative abundance of all barcodes within each well of each plate are measured using a single next-generation sequencing reaction.

PEXIL Results: Xenobiotic-metabolizing enzymes that convert the parent structures into lethal metabolites are evident because the DNA barcode associated with that enzyme is underrepresented within the sampled well. Unique combinations of xenobiotics and xenobiotic-metabolizing enzymes that lead to lethality are evident for the same reason.

PEXIL is used to reveal small molecules that are metabolically bioactivated into lethal products by enzymes, including cytochrome P450s: Through PEXIL technology, molecules that may selectively debilitate target organisms of interest are readily identified. Follow up in vitro or in vivo assays with the pathogen, parasite or pest of interest (and respective relevant non-targeted systems) is done to confirm the translation of hits to the targeted organism.

PEXIL is used to reveal lethal metabolites that are not reactive, which they themselves will be useful cidal agents. Metabolites that are generated by enzymes/P450s that kill cells are revealed by PEXIL and downstream analyses. These are purified and have utility on their own.

### Example 8: The Competitive CYPce/PEXIL Assay

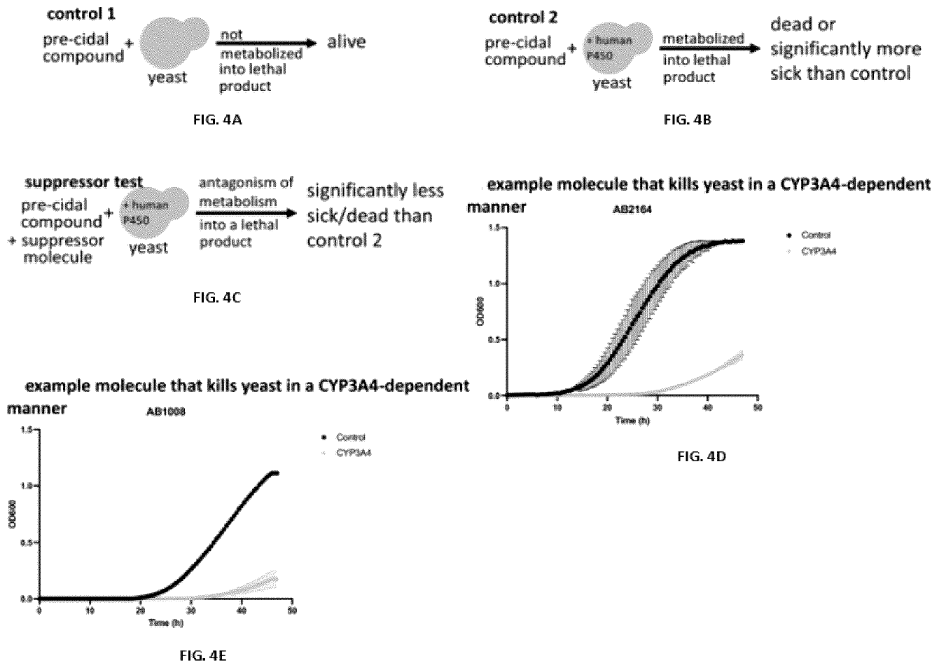

In some embodiments, CYPce/PEXIL is used as a competitive assay to identify substrates of P450s that may not be bioactivated (FIGS. 4A-4C). For example, molecules that kill yeast in a human CYP3A4-dependent manner have been identified (FIG. 4D-4E). In the background of one of these molecules (referred to here as the pre-cidal compound or PCC) incubated with yeast expressing CYP3A4, other compounds are screened for those that suppress the CYP3A4-dependent killing. Many of the hits that suppress killing/sickness in this background are molecules that compete with the PCC for CYP3A4, which are verified by analytical techniques (e.g. HPLC) to ensure that the PCC has not decreased in abundance in the cell. In this way, any molecule (e.g., molecules in drug pipelines or any other collection) can be screened for those that interact with the P450 of interest (and may therefore be a substrate for the P450 or be an inhibitor for the P450 or both).

The utility of Competitive CYPce/PEXIL to empirically identify compounds that interact with enzymes, including cytochrome P450s: Competitive CYPce and PEXIL is used to not only identify compounds that interact with CYP3A4, but is able to identify compounds that interact with a variety of compound-modifying enzymes, including any P450 and including all human P450s.

### Example 9: The Variants of Unknown Significance (VUS) CYPce/PEXIL Assay

Much variance of response to drugs in humans is due to differences in the ability of human P450s to metabolize the drugs. Some of that variance is due to P450 sequence polymorphisms. However, discerning which sequence variants have functional consequence and which do not is difficult. Being able to reliably determine whether a given sequence variant impacts P450 function has significant clinical implications.

CYPce, PEXIL, and competitive CYPce/PEXIL are exploited to determine the functional consequence of any P450 sequence variant of a compound-metabolizing enzyme/P450 by simply creating that sequence variant in the heterologously-expressed enzyme. This is done at the level of a single variant (competitive CYPce) or by creating a DNA-barcoded library of a single enzyme/P450, each strain of which encodes a distinct amino acid change in the relevant enzyme/P450. Using a pre-cidal compound (PCC) that is metabolized by the relevant enzyme/P450, the library of enzyme/P450 variants is then tested en masse for any change in sensitivity to the PCC, thereby revealing variants that impact the functionality of the enzyme/P450.

The current genetic backgrounds of the cells used in CYPce and PEXIL is modified to improve sensitivity of all assays described herein. systematic genetic (deletion, CRISPR, random mutagenesis etc) screens for single, double, or more complex backgrounds that increases the sensitivity of the assayed cell to the tests described herein. Theses genetic alterations may include, but are not limited to, increased accumulation of the pre-cidal compound (PCC) by any means; increased activity of the heterologously-expressed enzyme/P450 by any means; increased expression of the heterologously-expressed enzyme/P450 by any means; increased sensitivity of the cell to the resulting lethal metabolites by any means; increased accumulation of the resulting lethal metabolites by any means.

### Example 10: Exemplary Preparation Methods for Compounds of Formula I

The compounds of Formula I, wherein R¹ and R² are as defined in Formula I and n is 0, are prepared using the reaction conditions shown in Scheme 3:

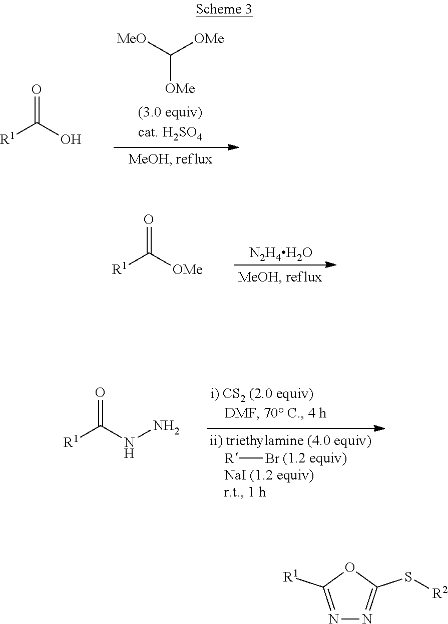

The compounds of Formula I, wherein R¹ and R² are as defined in Formula I and n is 1 or 2, are prepared using the reaction conditions shown in Scheme 4:

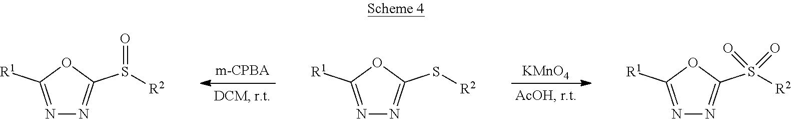

### Example 11: Dose-Response Analyses for Cyproside-3 and Structural Analogs

A structure-activity analysis was performed with 20 structural analogs of cyproside-3-1. Results are shown in FIG. 17. All but one had activity against at least one species of nematode that included C. elegans, D. dipsaci and M. hapla.

While the present disclosure has been described with reference to examples, it is to be understood that the scope of the claims should not be limited by the embodiments set forth in the examples but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present disclosure is found to be defined differently in a document incorporated herein by reference, the definition provided herein is to serve as the definition for the term.

TABLE 1

| | | GenBank Accession |
|---|---|---|
| P450 Name | Species | Number |
| CYP4C26 | Anopheles_gambiae | XP_001237336m |
| CYP4C27 | Anopheles_gambiae | XP_320022m |
| CYP4C35 | Anopheles_gambiae | XP_320018m |
| CYP4C36 | Anopheles_gambiae | XP_320019m |
| CYP4C25 | Anopheles_gambiae | XP_310940 |
| CYP4G16 | Anopheles_gambiae | XP_558699 |
| CYP4H17 | Anopheles_gambiae | XP_317096m |
| CYP4C28 | Anopheles_gambiae | XP_559040m |
| CYP4J5 | Anopheles_gambiae | XP_316098 |
| CYP4J10 | Anopheles_gambiae | XP_316099 |
| CYP4C37 | Anopheles_gambiae | XP_310941 |
| CYP4D15 | Anopheles_gambiae | XP_001237476 |
| CYP4D16 | Anopheles_gambiae | XP_003436121 |
| CYP4D17 | Anopheles_gambiae | XP_003436120 |
| CYP4D22 | Anopheles_gambiae | XP_312527 |
| CYP4AR1 | Anopheles_gambiae | XP_001237478 |
| CYP4G17 | Anopheles_gambiae | XP_555875 |
| CYP4H14 | Anopheles_gambiae | XP_321208 |
| CYP4H15 | Anopheles_gambiae | XP_001238447 |
| CYP4H16 | Anopheles_gambiae | XP_317098m |
| CYP325A2 | Anopheles_gambiae | XP_001230595 |
| CYP4H18 | Anopheles_gambiae | AGAP028019 |
| CYP4H19 | Anopheles_gambiae | XP_311064 |
| CYP4H24 | Anopheles_gambiae | XP_003436913 |
| CYP4H25 | Anopheles_gambiae | XP_003435957 |
| CYP4H26 | Anopheles_gambiae | XP_003435958 |
| CYP4H27 | Anopheles_gambiae | XP_557695m |
| CYP4J9 | Anopheles_gambiae | XP_316096 |
| CYP4K2 | Anopheles_gambiae | XP_001237479m |
| CYP4AA1 | Anopheles_gambiae | XP_313368 |
| CYP325A1 | Anopheles_gambiae | XP_565607 |
| CYP325A3 | Anopheles_gambiae | XP_307975 |
| CYP325B1 | Anopheles_gambiae | XP_565608 |
| CYP325C1 | Anopheles_gambiae | XP_307976 |
| CYP325C2 | Anopheles_gambiae | XP_307978 |
| CYP325C3 | Anopheles_gambiae | AGAP009696m |
| CYP325D2 | Anopheles_gambiae | XP_001687834m |
| CYP325E1 | Anopheles_gambiae | XP_001230597 |
| CYP325F1 | Anopheles_gambiae | XP_565621 |
| CYP325F2 | Anopheles_gambiae | XP_307986 |
| CYP325G1 | Anopheles_gambiae | XP_307987 |
| CYP325H1 | Anopheles_gambiae | XP_308056 |
| CYP325K1 | Anopheles_gambiae | XP_312384 |
| CYP15B1 | Anopheles_gambiae | XP_001237693 |
| CYP303A1 | Anopheles_gambiae | XP_319235m |
| CYP304B1 | Anopheles_gambiae | XP_001237487 |
| CYP304C1 | Anopheles_gambiae | XP_312753 |
| CYP305A2 | Anopheles_gambiae | XP_315675 |
| CYP305A3 | Anopheles_gambiae | XP_556304 |
| CYP305A4 | Anopheles_gambiae | XP_315679 |
| CYP306A1 | Anopheles_gambiae | XP_318345 |
| CYP307A2 | Anopheles_gambiae | XP_560266m |
| CYP307B1 | Anopheles_gambiae | XP_314781m |
| CYP6M1 | Anopheles_gambiae | XP_001237903 |
| CYP6M2 | Anopheles_gambiae | XP_317257 |
| CYP6M3 | Anopheles_gambiae | XP_317256 |
| CYP6M4 | Anopheles_gambiae | XP_001237902 |
| CYP6N2 | Anopheles_gambiae | XP_555257 |
| CYP6P1 | Anopheles_gambiae | XP_563271 |
| CYP6P2 | Anopheles_gambiae | XP_312047 |
| CYP6P3 | Anopheles_gambiae | XP_312050 |
| CYP6P4 | Anopheles_gambiae | XP_312048 |
| CYP6P5 | Anopheles_gambiae | XP_312049 |
| CYP6R1 | Anopheles_gambiae | XP_317263 |
| CYP6S1 | Anopheles_gambiae | XP_555259 |
| CYP6S2 | Anopheles_gambiae | XP_317264 |
| CYP6Y1 | Anopheles_gambiae | XP_317260 |
| CYP6Y2 | Anopheles_gambiae | XP_317261 |
| CYP6Z1 | Anopheles_gambiae | XP_317251 |
| CYP6Z2 | Anopheles_gambiae | XP_317252 |
| CYP6Z3 | Anopheles_gambiae | XP_555249 |
| CYP6Z4 | Anopheles_gambiae | XP_312016 |
| CYP6AA1 | Anopheles_gambiae | XP_312054 |
| CYP6AA2 | Anopheles_gambiae | XP_003436271 |
| CYP6AD1 | Anopheles_gambiae | XP_312046 |
| CYP6AF1 | Anopheles_gambiae | XP_309693 |
| CYP6AF2 | Anopheles_gambiae | XP_001688205 |
| CYP6AG1 | Anopheles_gambiae | XP_558354 |
| CYP6AG2 | Anopheles_gambiae | XP_003436449m |
| CYP6AH1 | Anopheles_gambiae | XP_308393 |
| CYP6AJ1 | Anopheles_gambiae | XP_563963m |
| CYP6AK1 | Anopheles_gambiae | XP_563971m |
| CYP9J3 | Anopheles_gambiae | XP_320246m |
| CYP9J4 | Anopheles_gambiae | XP_001238280 |
| CYP9J5 | Anopheles_gambiae | XP_551896 |
| CYP9K1 | Anopheles_gambiae | XP_316781 |
| CYP9L1 | Anopheles_gambiae | XP_320243 |
| CYP9L2 | Anopheles_gambiae | XP_001238278 |
| CYP9M1 | Anopheles_gambiae | XP_001688262m |
| CYP9M2 | Anopheles_gambiae | 001230703m |
| CYP329A1 | Anopheles_gambiae | XP_313263 |
| CYP314A1 | Anopheles_gambiae | XP_312515 |
| CYP12F1 | Anopheles_gambiae | XP_317439 |
| CYP12F2 | Anopheles_gambiae | XP_555443m |
| CYP12F3 | Anopheles_gambiae | XP_555444m |
| CYP12F4 | Anopheles_gambiae | XP_317441 |
| CYP49A1 | Anopheles_gambiae | XP_315789m |
| CYP301A1 | Anopheles_gambiae | XP_316138 |
| CYP302A1 | Anopheles_gambiae | XP_316034 |
| CYP315A1 | Anopheles_gambiae | XP_310837 |
| AaeL_AAEL017061 | Aedes aegypti | XP_011493257.1 |
| AaeL_AAEL017556 | Aedes aegypti | XP_011492960.1 |
| AAEL003349 | Aedes aegypti | XP_001656715.1 |
| 5564064 | Aedes aegypti | XP_001648376.1 |
| AAEL006824 | Aedes aegypti | XP_001658068.1 |
| CYP314A1 AAEL010946 | Aedes aegypti | XP_001661198.2 |
| CYP4G35 AAEL008345 | Aedes aegypti | XP_001659149.1 |
| CYP4C51 AAEL008018 | Aedes aegypti | XP_001658816.1 |
| 5569920 | Aedes aegypti | XP_001658815.1 |
| AAEL014618 | Aedes aegypti | XP_001649106.1 |
| CYP9J10 AAEL006798 | Aedes aegypti | XP_001652220.2 |
| CYP9J9 AAEL006793 | Aedes aegypti | XP_001652219.2 |
| AAEL014605 | Aedes aegypti | XP_001649108.1 |
| AAEL014614 | Aedes aegypti | XP_001649107.1 |
| CYP9J19 AAEL006810 | Aedes aegypti | XP_001652222.1 |
| CYP9J32 AAEL008846 | Aedes aegypti | XP_001653454.2 |
| CYP9J8 AAEL006811 | Aedes aegypti | XP_001652223.2 |
| 5579927 | Aedes aegypti | XP_001652216.1 |
| CYP9M7 AAEL001292 | Aedes aegypti | XP_001653043.2 |
| 5564758 | Aedes aegypti | XP_001649094.1 |
| 5564760 | Aedes aegypti | XP_001649093.1 |
| 5579928 | Aedes aegypti | XP_001652217.1 |
| CYP9J15 AAEL006795 | Aedes aegypti | XP_001652214.2 |
| AAEL014608 | Aedes aegypti | XP_001649104.1 |
| AAEL014604 | Aedes aegypti | XP_001649103.1 |
| CYP9J20 AAEL006814 | Aedes aegypti | XP_001652224.1 |
| CYP9J24 AAEL014613 | Aedes aegypti | XP_001649098.1 |
| CYP9J27 AAEL014607 | Aedes aegypti | XP_001649096.1 |
| 5564751 | Aedes aegypti | XP_001649095.1 |
| CYP307B1_a CYP307B1_b | Aedes aegypti | XP_001648198.1 |
| AAEL006875 AAEL014208 | | |
| 5579929 | Aedes aegypti | XP_001652218.2 |
| CYP9J27 AAEL014616 | Aedes aegypti | XP_001649109.1 |
| CYP9J27 | Aedes aegypti | XP_001649109.1 |
| CYP9M8 AAEL009591 | Aedes aegypti | XP_001660274.2 |
| CYP9J18 AAEL006804 | Aedes aegypti | XP_001652221.2 |
| CYP9J26 AAEL014609 | Aedes aegypti | XP_001649097.1 |
| AAEL014619 | Aedes aegypti | XP_001649101.1 |
| 5570872 | Aedes aegypti | XP_001659378.1 |
| CYP9J22 AAEL006802 | Aedes aegypti | XP_001652225.1 |
| CYP9J23 AAEL014615 | Aedes aegypti | XP_001649100.2 |
| 5565588 | Aedes aegypti | XP_001649970.1 |
| CYP9M6v2 5569822 | Aedes aegypti | XP_001653042.1 |
| CYP9M6 AAEL001312 | Aedes aegypti | XP_001653042.1 |
| CYP9M6v4 | Aedes aegypti | XP_001653042.1 |
| CYP4C52 AAEL008023 | Aedes aegypti | XP_001658817.1 |
| 5575453 | Aedes aegypti | XP_001661969.1 |
| 5575451 | Aedes aegypti | XP_001661968.1 |
| CYP9M4 AAEL001320 | Aedes aegypti | XP_001653040.1 |
| CYP307A1 AAEL009762 | Aedes aegypti | XP_001660339.2 |
| CYP9M9 AAEL001807 | Aedes aegypti | XP_001660386.2 |
| CYP9J7 AAEL014606 | Aedes aegypti | XP_001649099.1 |
| AAEL014891 | Aedes aegypti | XP_001649934.1 |
| 5578920 | Aedes aegypti | XP_001657206.1 |
| 5573117 | Aedes aegypti | XP_001660726.1 |
| AAEL014611 | Aedes aegypti | XP_001649105.1 |
| CYP12F6 AAEL002005 | Aedes aegypti | XP_001660728.2 |
| 5571532 | Aedes aegypti | XP_001653677.1 |
| 5564380 | Aedes aegypti | XP_001648725.1 |
| CYP12F7 AAEL002031 | Aedes aegypti | XP_001660729.2 |
| CYP325X4 AAEL005700 | Aedes aegypti | XP_001651330.2 |
| CYP325E3 AAEL000338 | Aedes aegypti | XP_001655571.1 |
| CYP301A1 AAEL014594 | Aedes aegypti | XP_001649083.2 |
| CYP325Z1 AAEL010273 | Aedes aegypti | XP_001660715.2 |
| AAEL001288 | Aedes aegypti | XP_001653041.1 |
| 5564381 | Aedes aegypti | XP_001648726.1 |
| CYP6CA1 AAEL014680 | Aedes aegypti | XP_001649310.1 |
| CYP329B1 AAEL003763 | Aedes aegypti | XP_001657205.1 |
| CYP325U1 AaeL_AAEL017215 | Aedes aegypti | XP_011493616.1 |
| 5564382 | Aedes aegypti | XP_001648727.1 |
| AAEL011463 | Aedes aegypti | XP_001661673.1 |
| CYP302A1 AAEL015655 | Aedes aegypti | XP_001647565.1 |
| CYP325X2 AAEL005696 | Aedes aegypti | XP_001651329.2 |
| CYP6AK1 AAEL004941 | Aedes aegypti | XP_001650095.2 |
| CYP4H29 AAEL007830 | Aedes aegypti | XP_001658673.1 |
| AAEL014830 | Aedes aegypti | XP_001649682.1 |
| 5565336 | Aedes aegypti | XP_001663751.1 |
| 5571531 | Aedes aegypti | XP_001653675.1 |
| CYP6AL3 AAEL009656 | Aedes aegypti | XP_001653903.1 |
| 5567035 | Aedes aegypti | XP_001651434.2 |
| CYP325K2 AAEL005771 | Aedes aegypti | XP_001651433.1 |
| 5578182 | Aedes aegypti | XP_001663753.1 |
| CYP4J14 AAEL013554 | Aedes aegypti | XP_001663752.1 |
| CYP12F8 AAEL006827 | Aedes aegypti | XP_001658065.1 |
| 5576028 | Aedes aegypti | XP_001655966.1 |
| 5571193 | Aedes aegypti | XP_001653509.1 |
| CYP6CD1 AAEL005006 | Aedes aegypti | XP_001650160.1 |
| CYP6P12 AAEL012491 | Aedes aegypti | XP_001662606.2 |
| 5565578 | Aedes aegypti | XP_001649932.1 |
| CYP325N1 AAEL012770 | Aedes aegypti | XP_001662864.2 |
| 5578646 | Aedes aegypti | XP_001663987.1 |
| 5566915 | Aedes aegypti | XP_001651328.1 |
| CYP325M5 AAEL011761 | Aedes aegypti | XP_001661896.2 |
| CYP325S1 AAEL000326 | Aedes aegypti | XP_001655567.2 |
| 110679581 5564964 | Aedes aegypti | XP_001649312.1 |
| CYP6F3 AAEL014684 | Aedes aegypti | XP_001649311.1 |
| 5579144 | Aedes aegypti | XP_001664239.2 |
| AAEL002872 | Aedes aegypti | XP_001656107.1 |
| 5573388 | Aedes aegypti | XP_001654558.1 |
| CYP6Y3 AAEL009132 | Aedes aegypti | XP_001653681.2 |
| CYP6AA5 AAEL012492 | Aedes aegypti | XP_001662604.1 |
| CYP325M4 AAEL011769 | Aedes aegypti | XP_001661897.1 |
| AAEL009018 | Aedes aegypti | XP_001659661.1 |
| 5569664 | Aedes aegypti | XP_001652928.1 |
| CYP6CB1 AAEL002046 | Aedes aegypti | XP_001654580.1 |
| CYP325M1 AAEL012773 | Aedes aegypti | XP_001662863.2 |
| CYP4D24 AAEL007815 | Aedes aegypti | XP_001652929.2 |
| 5569214 | Aedes aegypti | XP_001658387.1 |
| CYP4H28 AAEL003380 | Aedes aegypti | XP_001656766.2 |
| CYP4J17 AaeL_AAEL014019 | Aedes aegypti | XP_011493771.1 |
| CYP325N2 AAEL012762 | Aedes aegypti | XP_001662869.2 |
| CYP6BZ1 AAEL012494 | Aedes aegypti | XP_001662605.1 |
| CYP6AG5 AAEL006984 | Aedes aegypti | XP_001652485.2 |
| 5569663 | Aedes aegypti | XP_001652927.1 |
| CYP4D37 AAEL007795 | Aedes aegypti | XP_001652926.2 |
| CYP325R1 AAEL005775 | Aedes aegypti | XP_001651422.1 |
| 5576783 | Aedes aegypti | XP_001662870.1 |
| 5569659 | Aedes aegypti | XP_001652922.1 |
| 5575186 | Aedes aegypti | XP_001655568.1 |
| AAEL014924 | Aedes aegypti | XP_001649997.1 |
| CYP325G3 AAEL012772 | Aedes aegypti | XP_001662865.2 |
| CYP4AR2 AAEL010154 | Aedes aegypti | XP_001660657.1 |
| AAEL000325 | Aedes aegypti | XP_001655569.1 |
| 5571524 | Aedes aegypti | XP_001653676.1 |
| 5568653 | Aedes aegypti | XP_001652487.1 |
| 5568652 | Aedes aegypti | XP_001652486.1 |
| CYP6N17 AAEL010158 | Aedes aegypti | XP_001660663.2 |
| 5571549 | Aedes aegypti | XP_001653671.1 |
| 5569661 | Aedes aegypti | XP_001652925.2 |
| CYP306A1 AAEL004888 | Aedes aegypti | XP_001649971.2 |
| AAEL003890 | Aedes aegypti | XP_001664290.1 |
| 5575192 | Aedes aegypti | XP_001655570.2 |
| CYP6AG8 AAEL015654 | Aedes aegypti | XP_001647566.1 |
| 5571528 | Aedes aegypti | XP_001653673.1 |
| CYP325AA1 AAEL004012 | Aedes aegypti | XP_001648271.2 |
| CYP6N16 AAEL010151 | Aedes aegypti | XP_001660664.2 |
| CYP6M11 AAEL009127 | Aedes aegypti | XP_001653687.2 |
| CYP325Y3 AAEL006246 | Aedes aegypti | XP_001657610.2 |
| 5567662 | Aedes aegypti | XP_001657609.1 |
| AAEL002067 | Aedes aegypti | XP_001654600.1 |
| CYP305A6 AAEL002071 | Aedes aegypti | XP_001654599.1 |
| AAEL000340 | Aedes aegypti | XP_001655574.1 |
| 5576781 | Aedes aegypti | XP_001662868.2 |
| CYP6AG4 AAEL007010 | Aedes aegypti | XP_001652490.1 |
| 5568654 | Aedes aegypti | XP_001652488.1 |
| 5575901 | Aedes aegypti | XP_001662278.1 |
| 5571533 | Aedes aegypti | XP_001653678.1 |
| 5571526 | Aedes aegypti | XP_001653672.1 |
| 5569666 | Aedes aegypti | XP_001652930.1 |
| 5573422 | Aedes aegypti | XP_001654598.1 |
| AAEL014892 | Aedes aegypti | XP_001649933.1 |
| CYP325Q1 AAEL006044 | Aedes aegypti | XP_001651778.1 |
| CYP4H30 AAEL003399 | Aedes aegypti | XP_001656765.2 |
| CYP325M2 AAEL012769 | Aedes aegypti | XP_001662867.2 |
| CYP325Y2 AaeL_AAEL017165 | Aedes aegypti | XP_011493126.1 |
| CYP6CC1 AAEL014890 | Aedes aegypti | XP_001649935.2 |
| CYP6Z9 AAEL009129 | Aedes aegypti | XP_001653691.1 |
| 5571538 | Aedes aegypti | XP_001653683.1 |
| 23687717 | Aedes aegypti | XP_011492959.1 |
| 5571539 | Aedes aegypti | XP_001653686.1 |
| 5571529 | Aedes aegypti | XP_001653674.1 |
| 5571537 | Aedes aegypti | XP_001653682.1 |
| 23687959 | Aedes aegypti | XP_011493176.1 |
| 5575358 | Aedes aegypti | XP_001661895.1 |
| 5571543 | Aedes aegypti | XP_001653690.1 |
| 5571541 | Aedes aegypti | XP_001653688.1 |
| 5579336 | Aedes aegypti | XP_001651779.1 |
| AAEL014612 | Aedes aegypti | XP_001649102.1 |
| CYP325T2 AAEL012761 | Aedes aegypti | XP_001662866.1 |
| CYP315A1 AAEL011850 | Aedes aegypti | XP_001661987.2 |
| CYP325V1 AaeL_AAEL017136 | Aedes aegypti | XP_011493614.1 |
| AAEL014829 | Aedes aegypti | XP_001649683.1 |
| AAEL015663 | Aedes aegypti | XP_001647559.1 |
| CYP307D1 | Ixodes_scapularis | XP_029828623 |
| CYP18C1 | Ixodes_scapularis | XP_029844575 |
| CYP3001N5 | Ixodes_scapularis | XP_029831820 |
| CYP3001N6 | Ixodes_scapularis | XP_029831821mN |
| CYP3001N7 | Ixodes_scapularis | XP_029831821mC |
| CYP3001N4 | Ixodes_scapularis | XP_029831822 |
| CYP3001N1 | Ixodes_scapularis | XP_029831816 |
| CYP3001P1 | Ixodes_scapularis | XP_029826022 |
| CYP3001P2 | Ixodes_scapularis | XP_002415169 |
| CYP3001Q2 | Ixodes_scapularis | XP_029826019 |
| CYP3001Q3 | Ixodes_scapularis | XP_029833587 |
| CYP3001Q4 | Ixodes_scapularis | XP_029826596m |
| CYP3002A1 | Ixodes_scapularis | XP_029851587 |
| CYP3002A3 | Ixodes_scapularis | XP_029851569 |
| CYP3002A2 | Ixodes_scapularis | XP_029851585 |
| CYP3002A4 | Ixodes_scapularis | XP_029851493 |
| CYP3001C2 | Ixodes_scapularis | XP_029826080m |
| CYP3001M4 | Ixodes_scapularis | XP_029824045 |
| CYP3003A3 | Ixodes_scapularis | XP_029845163 |
| CYP3003A4 | Ixodes_scapularis | XP_002400107 |
| CYP3003A5 | Ixodes_scapularis | XP_029845164 |
| CYP3003A6 | Ixodes_scapularis | XP_029845165 |
| CYP3003A8 | Ixodes_scapularis | XP_002400111 |
| CYP3003A9 | Ixodes_scapularis | XP_029845167 |
| CYP3001AH1 | Ixodes_scapularis | XP_029842302 |
| CYP3003A10 | Ixodes_scapularis | XP_029846882 |
| CYP3003A11 | Ixodes_scapularis | XP_029849595 |
| CYP3001P4 | Ixodes_scapularis | XP_029833956 |
| CYP3001M5 | Ixodes_scapularis | XP_029834954 |
| CYP3001M6 | Ixodes_scapularis | XP_029831721 |
| CYP3003A13 | Ixodes_scapularis | XP_029845091 |
| CYP3003A12 | Ixodes_scapularis | XP_029845059 |
| CYP3259B1 | Ixodes_scapularis | XP_029822576 |
| CYP3001E1 | Ixodes_scapularis | XP_029838890m |
| CYP3001G1 | Ixodes_scapularis | XP_029831815 |
| CYP3001H1 | Ixodes_scapularis | XP_029831819 |
| CYP3001K1 | Ixodes_scapularis | XP_029832961 |
| CYP3001C1 | Ixodes_scapularis | XP_029826089m |
| CYP3001M2 | Ixodes_scapularis | XP_029832670 |
| CYP3001A1 | Ixodes_scapularis | XP_029848055m |
| CYP3001B1 | Ixodes_scapularis | XP_002402171 |
| CYP3001B3 | Ixodes_scapularis | XP_029848629mincN |
| CYP3001B4 | Ixodes_scapularis | XP_029848628 |
| CYP20R1 | Ixodes_scapularis | XP_002404438 |
| CYP3011A1 | Ixodes_scapularis | XP_002435754 |
| CYP3004A1v1 | Ixodes_scapularis | XP_029848772 |
| CYP3004A2v1 | Ixodes_scapularis | XP_002401737 |
| CYP3011A2v2a | Ixodes_scapularis | XP_029851214 |
| CYP3011A2v2b | Ixodes_scapularis | XP_029831512 |
| CYP3005A6v1 | Ixodes_scapularis | XP_029842880 |
| CYP3005A7v1 | Ixodes_scapularis | XP_002413514 |
| CYP3006A1v1 | Ixodes_scapularis | XP_029851283 |
| CYP41C7 | Ixodes_scapularis | XP_029822945m |
| CYP3006A1v2 | Ixodes_scapularis | XP_002405300m |
| CYP3009B1 | Ixodes_scapularis | XP_029823408 |
| CYP3009A1 | Ixodes_scapularis | XP_002407452 |
| CYP3009A2 | Ixodes_scapularis | XP_029823442 |
| CYP3009A3 | Ixodes_scapularis | XP_029823444m |
| CYP3009A4 | Ixodes_scapularis | XP_029823441 |
| CYP3009A5 | Ixodes_scapularis | XP_002407455 |
| CYP3009A | Ixodes_scapularis | XP_029823445 |
| CYP3009A6 | Ixodes_scapularis | XP_029823404 |
| CYP3009A8 | Ixodes_scapularis | XP_029823402 |
| CYP3009A9 | Ixodes_scapularis | XP_029823406 |
| CYP3009A10 | Ixodes_scapularis | XP_029823407mN |
| CYP3009A11 | Ixodes_scapularis | XP_029823407mC |
| CYP3009C1 | Ixodes_scapularis | XP_029823413 |
| CYP3009D4 | Ixodes_scapularis | XP_029823432 |
| CYP3009D6 | Ixodes_scapularis | XP_029823458 |
| CYP3009D1 | Ixodes_scapularis | XP_029823414 |
| CYP3009D2 | Ixodes_scapularis | XP_029823415 |
| CYP3009C2 | Ixodes_scapularis | XP_029823832 |
| CYP41C1 | Ixodes_scapularis | XP_029831758mN |
| CYP41C2 | Ixodes_scapularis | XP_029831758mC |
| CYP41A2v1 | Ixodes_scapularis | XP_029831771 |
| CYP41U1v1 | Ixodes_scapularis | XP_029831765 |
| CYP3006H1 | Ixodes_scapularis | XP_029842190 |
| CYP3006G3 | Ixodes_scapularis | XP_029843294 |
| CYP3006G6 | Ixodes_scapularis | XP_029843192 |
| CYP3006G1 | Ixodes_scapularis | XP_029843301m |
| CYP3006G2 | Ixodes_scapularis | XP_029843205m |
| CYP3006G8 | Ixodes_scapularis | XP_029843206m |
| CYP41C14 | Ixodes_scapularis | XP_029822041 |
| CYP3005A20v1 | Ixodes_scapularis | XP_029840517 |
| CYP3005A21 | Ixodes_scapularis | XP_002433441 |
| CYP3005A18 | Ixodes_scapularis | XP_029840521 |
| CYP3005A19 | Ixodes_scapularis | XP_029840523 |
| CYP3009A12 | Ixodes_scapularis | XP_029845645 |
| CYP41A2v2 | Ixodes_scapularis | XP_029838728mN |
| CYP41U1v2 | Ixodes_scapularis | XP_029838728mC |
| CYP41C16 | Ixodes_scapularis | XP_029838570m |
| CYP41C18a | Ixodes_scapularis | XP_029838566 |
| CYP41C17 | Ixodes_scapularis | XP_029844000 |
| CYP3005A6v2 | Ixodes_scapularis | XP_029843930 |
| CYP3005A7v2 | Ixodes_scapularis | XP_029843926 |
| CYP3005A17 | Ixodes_scapularis | XP_029843920m |
| CYP3005A14 | Ixodes_scapularis | XP_002399296 |
| CYP41C19 | Ixodes_scapularis | XP_029824043 |
| CYP3004C4 | Ixodes_scapularis | XP_029846165 |
| CYP3005A10 | Ixodes_scapularis | XP_029850272 |
| CYP3005A1 | Ixodes_scapularis | XP_029835894 |
| CYP3005A3 | Ixodes_scapularis | XP_029835880 |
| CYP3005A11 | Ixodes_scapularis | XP_029832847 |
| CYP3005A22 | Ixodes_scapularis | XP_029827583 |
| CYP3005A12v1 | Ixodes_scapularis | XP_029827572 |
| CYP3005A9v1 | Ixodes_scapularis | XP_029827397 |
| CYP3005A16 | Ixodes_scapularis | XP_029840516 |
| CYP3005A9v2 | Ixodes_scapularis | XP_029835004 |
| CYP3005A12v2 | Ixodes_scapularis | XP_029841022 |
| CYP3005A20v2 | Ixodes_scapularis | XP_029834782 |
| CYP41C9v1 | Ixodes_scapularis | XP_029825411 |
| CYP3004A1v2 | Ixodes_scapularis | XP_029828783 |
| CYP3004A2v2 | Ixodes_scapularis | XP_029828782 |
| CYP41C15 | Ixodes_scapularis | XP_029828197 |
| CYP3011A3 | Ixodes_scapularis | XP_029831534 |
| CYP3004C1 | Ixodes_scapularis | XP_029828446 |
| CYP3010A1 | Ixodes_scapularis | XP_029851667 |
| CYP41C13 | Ixodes_scapularis | XP_029851196 |
| CYP3004A1 | Ixodes_scapularis | XP_029831581 |
| CYP3004A5 | Ixodes_scapularis | XP_029831587m |
| CYP3004A6 | Ixodes_scapularis | XP_029831582 |
| CYP3004A7 | Ixodes_scapularis | XP_029831580 |
| CYP3004A8 | Ixodes_scapularis | XP_029831577 |
| CYP3004A3 | Ixodes_scapularis | XP_029831578 |
| CYP3004A9 | Ixodes_scapularis | XP_029831579 |
| CYP3004C2 | Ixodes_scapularis | XP_029836010 |
| CYP41C20 | Ixodes_scapularis | XP_029832601 |
| CYP41C6 | Ixodes_scapularis | XP_029832610mN |
| CYP41C21 | Ixodes_scapularis | XP_029832610mC |
| CYP41C22 | Ixodes_scapularis | XP_029832604 |
| CYP41C5 | Ixodes_scapularis | XP_029832599 |
| CYP3006E1 | Ixodes_scapularis | XP_029851126 |
| CYP3009A14 | Ixodes_scapularis | XP_029844812 |
| CYP41C9v2 | Ixodes_scapularis | XP_029831791 |
| CYP41C10 | Ixodes_scapularis | XP_029849822mN |
| CYP41C11 | Ixodes_scapularis | XP_029849822mC |
| CYP41C18b | Ixodes_scapularis | XP_029831740 |
| CYP3006G4 | Ixodes_scapularis | XP_029843194 |
| CYP3006G9 | Ixodes_scapularis | XP_029843304 |
| CYP41C23 | Ixodes_scapularis | XP_029847504 |
| CYP41B1 | Ixodes_scapularis | XP_029847199 |
| CYP3009B2 | Ixodes_scapularis | XP_029843213 |
| CYP41C12 | Ixodes_scapularis | XP_029833208 |
| CYP41C3 | Ixodes_scapularis | XP_029835816 |
| CYP3006F1 | Ixodes_scapularis | XP_029843195 |
| CYP3006G5 | Ixodes_scapularis | XP_029838649m |
| CYP3004A10 | Ixodes_scapularis | XP_029841861 |
| CYP3004B1 | Ixodes_scapularis | XP_029841841mC |
| CYP3004D2 | Ixodes_scapularis | XP_029841842 |
| CYP3004D1 | Ixodes_scapularis | XP_029841841mN |
| CYP4W5v1 | Ixodes_scapularis | XP_029845465 |
| CYP4W8v1 | Ixodes_scapularis | XP_029845468 |
| CYP4W2 | Ixodes_scapularis | XP_029845471 |
| CYP319A4v2 | Ixodes_scapularis | XP_029847575 |
| CYP4W5v2 | Ixodes_scapularis | XP_029847581 |
| CYP4W3v2 | Ixodes_scapularis | XP_029847582 |
| CYP4W8v2 | Ixodes_scapularis | XP_029847584 |
| CYP319A3 | Ixodes_scapularis | XP_029850544 |
| CYP4DP2 | Ixodes_scapularis | XP_029850547 |
| CYP4DS1 | Ixodes_scapularis | XP_029829448 |
| CYP4DS5 | Ixodes_scapularis | XP_002405700 |
| CYP4DS12 | Ixodes_scapularis | XP_029851272m |
| CYP319A4v1 | Ixodes_scapularis | XP_029849473 |
| CYP319A4v3 | Ixodes_scapularis | XP_029845458 |
| CYP4DS4 | Ixodes_scapularis | XP_029842105mC |
| CYP4W3v1 | Ixodes_scapularis | XP_029845466 |
| CYP4DS8v1 | Ixodes_scapularis | XP_029848209 |
| CYP4DS8v2 | Ixodes_scapularis | XP_029848811 |
| CYP4DP1 | Ixodes_scapularis | XP_029850540 |
| CYP319A5 | Ixodes_scapularis | XP_029850533mN |
| CYP319A7 | Ixodes_scapularis | XP_029850533mC |
| CYP319A6 | Ixodes_scapularis | XP_029850543 |
| CYP4W4 | Ixodes_scapularis | XP_029850546 |
| CYP4DL2 | Ixodes_scapularis | XP_029850537mC |
| CYP4DL1 | Ixodes_scapularis | XP_002412998 |
| CYP4DM2 | Ixodes_scapularis | XP_029850538 |
| CYP4DM1 | Ixodes_scapularis | XP_029850539m |
| CYP4DM3 | Ixodes_scapularis | XP_029850548m |
| CYP4DL5v1 | Ixodes_scapularis | XP_029850550 |
| CYP4DL4 | Ixodes_scapularis | XP_002404872 |
| CYP4DL6 | Ixodes_scapularis | XP_029850551 |
| CYP4DS9 | Ixodes_scapularis | XP_029832448 |
| CYP4DS11 | Ixodes_scapularis | XP_029832447 |
| CYP4DS6 | Ixodes_scapularis | XP_029832451 |
| CYP4DQ1 | Ixodes_scapularis | XP_029830564 |
| CYP4DR1v1a | Ixodes_scapularis | XP_029851273 |
| CYP4DS10v1 | Ixodes_scapularis | XP_002408834mN |
| CYP4DS13 | Ixodes_scapularis | XP_029825022m |
| CYP4DS14 | Ixodes_scapularis | XP_029825020 |
| CYP4W7 | Ixodes_scapularis | XP_029825032 |
| CYP4DM4 | Ixodes_scapularis | XP_029845500m |
| CYP4DR1v1b | Ixodes_scapularis | XP_029842104m |
| CYP4DS10v2 | Ixodes_scapularis | XP_029842105mN |
| CYP4DL3 | Ixodes_scapularis | XP_029827749m |
| CYP4DL5v2 | Ixodes_scapularis | XP_029826974 |
| CYP315A1 | Ixodes_scapularis | XP_002411841 |
| CYP314A1 | Ixodes_scapularis | XP_002401077 |
| CYP302A1 | Ixodes_scapularis | XP_029829236 |
| CYP3012A1 | Ixodes_scapularis | XP_029836282 |

### REFERENCES

Binan, L., Drobetsky, E. A., and Costantino, S. (2019). Exploiting Molecular Barcodes in High-Throughput Cellular Assays. SLAS Technol 24, 298-307.
Durairaj, P., Fan, L., Du, W., Ahmad, S., Mebrahtu, D., Sharma, S., Ashraf, R. A., Liu, J., Liu, Q., and Bureik, M. (2019). Functional expression and activity screening of all human cytochrome P450 enzymes in fission yeast. FEBS Lett 593, 1372-1380.
Hausjell, J., Halbwirth, H., and Spadiut, O. (2018). Recombinant production of eukaryotic cytochrome P450s in microbial cell factories. Biosci Rep 38.
McMahon, K. W., Manukyan, A., Dungrawala, H., Montgomery, M., Nordstrom, B., Wright, J., Abraham, L., and Schneider, B. L. (2011). FASTA barcodes: a simple method for the identification of yeast ORF deletions. Yeast 28, 661-671.
Rautio, J., Meanwell, N. A., Di, L., and Hageman, M. J. (2018). The expanding role of prodrugs in contemporary drug design and development. Nat Rev Drug Discov 17, 559-587.

1. An assay to identify xenobiotics that when bioactivated can debilitate a target organism comprising:
a) heterologously expressing a xenobiotic metabolizing enzyme from the target organism in a yeast;
b) exposing the yeast to a test molecule; and
c) comparing the viability of the yeast in b) with control yeast that do not heterologously express the xenobiotic metabolizing enzyme from the target organism;
wherein if the viability of the yeast is less than the control, then the test molecule is a candidate xenobiotic for debilitating the target organism.

2. The assay of claim 1, wherein the xenobiotic metabolizing enzyme is a cytochrome P450 enzyme, an esterase, an alkaline phosphatase, an amidase, a phospholipase, a paraoxonase, a carboxymethylenebutenolidase or a hydrolase from the target organism.

3. The assay of claim 1, wherein the coding sequence of the xenobiotic metabolizing enzyme is tagged with a sequence that encodes another protein to enable tracking of the enzyme's expression level and sub-cellular localization, optionally wherein the coding sequence of the xenobiotic metabolizing enzyme is codon-optimized.

4. The assay of claim 1, wherein the xenobiotic metabolizing enzyme is contained in a plasmid that allows for controlled expression in yeast or is integrated into the yeast genome.

5. The assay of claim 1, wherein the yeast comprises a plasmid comprising a unique DNA barcode, or the yeast comprises a unique DNA barcode integrated into the yeast genome.

6. The assay of claim 1, further comprising heterologously expressing xenobiotic metabolizing enzyme co-factors that are required for improved activity of the xenobiotic metabolizing enzyme, optionally wherein the xenobiotic metabolizing co-factors are expressed from the same transgene as the xenobiotic metabolizing enzyme or the xenobiotic metabolizing co-factors are expressed from a separate transgene than the xenobiotic metabolizing enzyme.

7. The assay of claim 1, wherein the target organism is a pest, pathogen or parasite, optionally target organism is a nematode or an insect.

8. The assay of claim 1, wherein the assay further comprises testing the candidate xenobiotic with a yeast heterologously expressing a xenobiotic metabolizing enzyme of a non-target organism, and testing viability of the yeast compared to a control not expressing the xenobiotic metabolizing enzyme of the non-target organism, wherein the candidate xenobiotic is selective against the target organism if the viability of the yeast heterologously expressing the xenobiotic enzyme of the non-target organism is similar to the control, optionally the non-target organism is a human, pet, livestock, pollinator, bird, fish, vertebrate or plant.

9. A library comprising yeast strains, wherein each yeast strain heterologously expresses a different xenobiotic metabolizing enzyme from one or more target organisms, and wherein each yeast strain contains a unique DNA barcode in a plasmid or integrated into a fixed or random location within the yeast genome.

10. The library of claim 9, wherein the xenobiotic metabolizing enzyme in each yeast strain is contained in a plasmid or is integrated into the yeast genome and/or the target organism is a pest, pathogen or parasite, optionally the target organism is a nematode or an insect.

11. A library comprising yeast strains, wherein each yeast strain heterologously expresses a different xenobiotic metabolizing enzyme from one or more non-target organisms, wherein each yeast strain contains a unique DNA barcode in a plasmid or integrated into a fixed or random location within the yeast genome.

12. The library of claim 11, wherein the xenobiotic metabolizing enzyme in each yeast strain is contained in a plasmid or is integrated into the yeast genome and/or wherein the non-target organism is a human, pet, livestock, pollinator, bird, fish, vertebrate or plant.

13. A method of screening libraries with xenobiotics to identify a xenobiotic that when bioactivated debilitates a target organism, comprising
a) pooling the yeast strains of the library of claim 9;
b) depositing the pooled library into wells of a multiwell plate;
c) exposing each well of the multiwell plate to a test xenobiotic;
d) incubating the plate of c) to allow the yeast to grow;
e) collecting samples from d) and isolating DNA; and
f) detecting the relative abundance of the unique DNA barcodes;
wherein a decrease in the abundance of a particular unique DNA barcode indicates that the xenobiotic metabolizing enzyme in the yeast strain containing that DNA barcode bioactivates the test xenobiotic and indicates that it is able to debilitate the target organism.

14. The method of claim 13, further comprising
g) pooling yeast strains of a library comprising the yeast strains, wherein each yeast strain heterologously expresses a different xenobiotic metabolizing enzyme from one or more non-target organisms, wherein each yeast strain contains a unique DNA barcode in a plasmid or integrated into a fixed or random location within the yeast genome,
h) depositing the pooled library of g) into wells of a multiwell plate;
i) exposing each well of the multiwell plate to the test xenobiotic;
j) incubating the plate of i) to allow the yeast to grow;
k) collecting samples from j) and isolating DNA; and
l) detecting the relative abundance of the unique DNA barcodes;
wherein the test xenobiotic is selective if it does not decrease the abundance of a particular DNA barcode of the yeast strains of the library of g), optionally steps g) to l) are performed in parallel with steps a) to f).

15. A method of treating or preventing a nematode infection comprising administering, to a subject in need thereof, an effective amount of a compound of Formula I, or a solvate thereof: 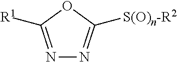
wherein:
R¹ is selected from phenyl and 6-membered heteroaryl, both of which are optionally substituted with one to three substituents independently selected from halo, NO₂, C₁₋₆alkyl, OC₁₋₆alkyl, C(O)C₁₋₆alkyl, OC(O)C₁₋₆alkyl and C(O)OC₁₋₆alkyl, wherein each alkyl group is optionally fluorosubstituted;
R² is selected from CF₃, C₁₋₆alkyl and C₁₋₆alkenyl; and
n is 0, 1 or 2.

16. The method of claim 15, wherein R¹ is phenyl optionally substituted with one to three substituents independently selected from Cl, Br, F, NO₂, C₁₋₄alkyl, OC₁₋₄alkyl, C(O)C₁₋₄alkyl, OC(O)C₁₋₄alkyl and C(O)OC₁₋₄alkyl, wherein each alkyl group is optionally fluorosubstituted; or
R¹ is a 6-membered heteroaryl optionally substituted with one to three substituents independently selected from Cl, Br, F, NO₂, C₁₋₄alkyl, OC₁₋₄alkyl, C(O)C₁₋₄alkyl, OC(O)C₁₋₄alkyl and C(O)OC₁₋₄alkyl;
R² is selected from C₁₋₄alkyl and C₁₋₄alkenyl, and
n is 1.

17. The method of claim 15, wherein the compound of Formula I is selected from: 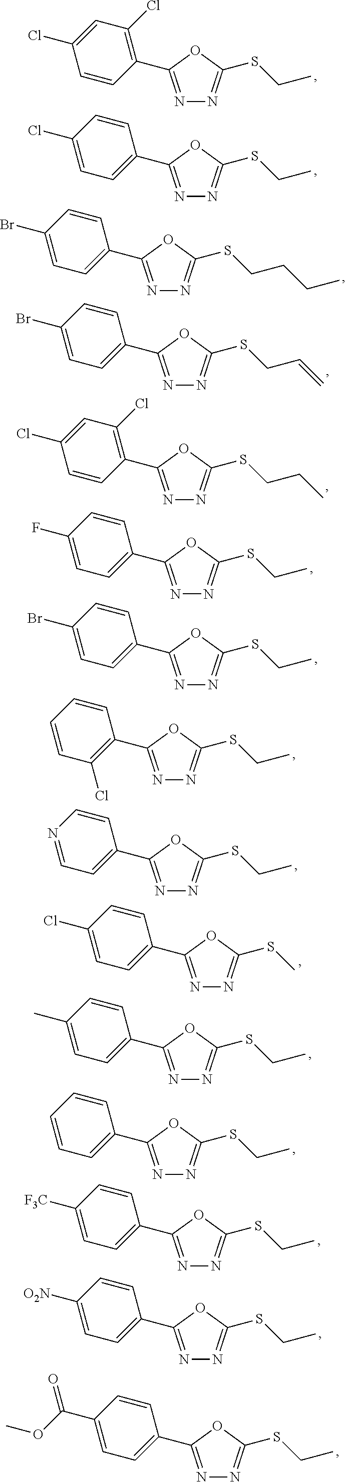 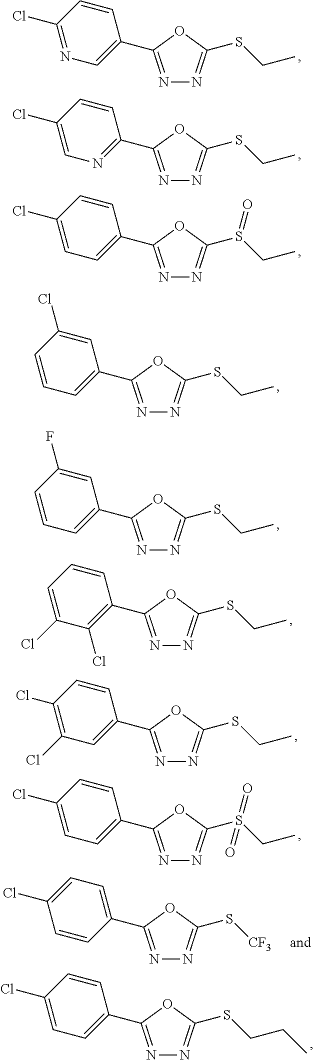
or a solvate thereof.

18. The method of claim 15, wherein the infection is an infection of a nematode of a species selected from members of the genuses Pratylenchus, Heterodera, Meloidogyne, Ditylenchus or Pratylenchus, Cooperia and Haemonchus, including nematodes of the species selected from Heterodera glycines, Globodera pallida, Meloidogyne javanica, Meloidogyne incognita, Meloidogyne arenaria, Radopholus similis, Longidorus elongatus, Meloidogyne hapla, Ditylenchus dipsaci, Pratylenchus penetrans, Cooperia oncophora, and Haemonchus contortus.

19. The method of claim 15, wherein the subject is selected from a human, a mammal, a bird, a plant, a seed, and soil.

20. A compound selected from: 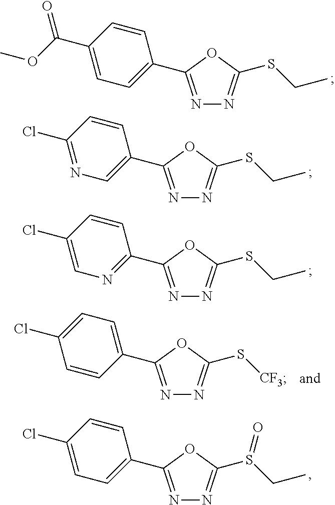
or a solvate thereof.
